(19) United States
(12) Patent Application Publication
(10) Pub. No.: US 2026/0160766 A1
(43) Pub. Date: Jun. 11, 2026

(54) METHODS FOR THE DETECTION AND TREATMENT OF LUNG CANCER

(71) Applicant: Board of Regents, The University of Texas System, Austin, TX (US)

(72) Inventors: Ehsan IRAJIZAD, Houston, TX (US); Johannes Francois FAHRMANN, Houston, TX (US); Samir HANASH, Houston, TX (US); Edwin Justin OSTRIN, Houston, TX (US)

(21) Appl. No.: 19/404,239

(22) Filed: Dec. 1, 2025

Related U.S. Application Data
(63) Continuation of application No. PCT/US2024/031238, filed on May 28, 2024.
(60) Provisional application No. 63/505,148, filed on May 31, 2023.

(51) Int. Cl. G01N 33/5752; G01N 33/575; G01N 33/68; G16B 20/00; G16B 40/20

(52) U.S. Cl. CPC G01N 33/5752; G01N 33/57585; G01N 33/6851; G16B 20/00; G16B 40/20

(57) ABSTRACT

Provided are methods for identifying individuals at high risk of lung cancer death.

This application is a bypass continuation of international application no. PCT/US2024/031238, filed May 28, 2024, which claims the benefit of priority of U.S. provisional application No. 63/505,148, filed May 31, 2023, the contents of which are incorporated by reference as if written herein in their entirety.

This invention was made with government support under CA194733 awarded by the National Institutes of Health. The government has certain rights in the invention.

Lung cancer is the most prevalent cancer in the United States, with a five-year survival rate of less than 15%. Recently, therapies for lung cancer have begun to transition from a limited selection of radiation, folate metabolism, platinum-based drugs, and taxane-based drugs to more targeted treatments that require histological characterization of the tumor and/or the presence or absence of key biomarker or therapeutic target proteins.

Data from the National Lung Screening Trial (NLST) suggests that yearly screening of high-risk current and ex-smokers with thoracic low-dose computed tomography (LDCT) has been shown to reduce mortality due to lung cancer by 20%. In 2021, the United States Preventive Service Task Force (USPSTF) expanded the eligibility for LDCT screening and now recommends annual screening for lung cancer with LDCT for adults aged 50-80 years who have a smoking history greater than 20 pack-years and either currently smoke or have quit within the past 15 years. However, there are several negative aspects associated with CT screening in terms of morbidity, including over-diagnosis, false positives, over-treatment, and financial costs.

There is an abundance of literature on lung cancer risk prediction on the potential benefit of supplementing the USPSTF screening criteria with a risk-based model when identifying subjects for CT-screening. For instance, recently it was estimated that 20% of additional lung cancer deaths could be avoided by using a screening criterion based on individual risk assessment. The information required to utilize risk-prediction tools could be readily ascertained by a general practitioner—or potentially self-assessed using an online risk-calculator—making future lung cancer screening programs likely to implement such tools when assessing screening eligibility.

One such tool would be an individual-level risk-based screening criteria that accurately estimates the risk of lung cancer within the near future (e.g., 1-3 years) for a given subject. Several risk prediction models have been published that rely on demographic data (age, sex, etc.) and risk factor data from questionnaires, such as PLCOₘ₂₀₁₂ and the Liverpool Lung Project (LLP). Elevated levels of protein biomarkers have also been found to serve as useful predictors of the risk of developing lung cancer. A novel blood-based four-marker protein panel comprising or consisting of pro-surfactant protein B (pro-SFTPB), Mucin 16 (CA125), carcinoembryonic antigen (CEA), and cytokeratin-19 fragment (CYFRA21-1) is described in U.S. Ser. No. 16/484,177, the contents of which are hereby incorporated by reference in their entirety. The use of this panel in combination with PLCOₘ₂₀₁₂ has been found to significantly improve lung cancer risk assessment compared to former and current USPSFT criteria for lung cancer screening. As cellular and systemic metabolic adaptations occur from the earliest phases of cancer development, there is evidence that further improvements can be made through the identification and use of small molecule metabolites as cancer biomarkers.

Accordingly, a need exists for a method or test to aid the detection of lung cancer death.

### SUMMARY

Provided herein is a method of method of predicting the prognosis of an individual having a lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
wherein the combined model score indicates the prognosis of the individual with respect to time to progression and overall survival.

Also provided is a method of predicting disease outcome in an individual having lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having an increased risk of a poor disease outcome when the combined model score is above is above a pre-defined risk threshold.

Also provided is a method for identifying an individual at high risk of lung cancer death, comprising:
determining a combined model score using a logistic regression with a PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having a high risk of lung cancer death when the combined model score is above is above a pre-defined risk threshold.

### BRIEF DESCRIPTION OF THE DRAWINGS

FIG. 1 depicts the predictive performance of the 4MP, PLCOₘ₂₀₁₂, and the combined 4MP+PLCOₘ₂₀₁₂ model for predicting lung cancer-specific mortality. Case sera collected within 1 year of diagnosis and all non-case sera were considered. Nodes show corresponding sensitivity and specificity based on USPSTF2013 or 2021 criteria.

FIG. 2 depicts the predictive performance of the 4MP, PLCOₘ₂₀₁₂, and the combined 4MP+PLCOₘ₂₀₁₂ models for predicting 1-year lung cancer-specific mortality.

FIG. 3 depicts the predictive performance of the 4MP, PLCOₘ₂₀₁₂, and the combined 4MP+PLCOₘ₂₀₁₂ models for predicting 6-year lung cancer-specific mortality.

FIG. 4 depicts the predictive performance of the 4MP, PLCOₘ₂₀₁₂, and the combined 4MP+PLCOₘ₂₀₁₂ models for predicting lung cancer-specific mortality among ever-smokers with a <10 PY smoking history.

FIG. 5 depicts the predictive performance of the 4MP, PLCOₘ₂₀₁₂, and the combined 4MP+PLCOₘ₂₀₁₂ models for predicting lung cancer-specific mortality among ever-smokers with a ≥10PY smoking history.

FIG. 6 depicts the predictive performance of the 4MP, PLCOₘ₂₀₁₂, and the combined 4MP+PLCOₘ₂₀₁₂ models for predicting lung cancer-specific mortality among unique cases and non-cases.

FIG. 7 depicts the cumulative incidence plot for lung cancer death for individuals who were test-positive or test-negative based on 4MP+PLCOₘ₂₀₁₂ scores ≥ or <the 1.0% and 1.7% 6-year risk thresholds. Analyses were based cases diagnosed within 1 year of blood draw and all non-case participants with ≥10 PYS smoking history.

FIG. 8 depicts the cumulative incidence plot for lung cancer death amongst all asymptotic individuals who were test-positive or test-negative based on 4MP scores at a 1.0% 6-year risk threshold. Analyses were based on cases with ≥10 PYS smoking history and all lung cancer cases are within 1 year of clinical diagnosis with cancer.

FIG. 9 depicts the cumulative incidence plot for lung cancer death amongst all asymptotic individuals who were test-positive or test-negative based on 4MP scores at a 1.7% 6-year risk threshold.

FIG. 10 depicts the cumulative incidence plot for lung cancer death amongst all asymptotic individuals who were test-positive or test-negative based on PLCOₘ₂₀₁₂ scores at a 1% 6-year risk threshold.

FIG. 11 depicts the cumulative incidence plot for lung cancer death amongst all asymptotic individuals who were test-positive or test-negative based on PLCOₘ₂₀₁₂ scores at a 1.7% 6-year risk threshold.

FIG. 12 depicts the cumulative incidence plot for lung cancer death and non-lung cancer death diagnosed within 1 years of blood draw who were test-positive or test-negative based on 4MP+PLCOₘ₂₀₁₂ scores at a 1.0% 6-year risk threshold. Analyses were based on cases with ≥10 PYS smoking history.

FIG. 13 depicts the cumulative incidence plot for lung cancer death and non-lung cancer death diagnosed within 1 years of blood draw who were test-positive or test-negative based on 4MP+PLCOₘ₂₀₁₂ scores at a 1.7% 6-year risk threshold.

FIG. 14 depicts the cumulative incidence plot for lung cancer death amongst all asymptotic individuals who were test-positive or test-negative based on 4MP plus PLCOₘ₂₀₁₂ scores at a 1.0% 6-year risk threshold. Analyses were based on cases with ≥10 PYS smoking history and all lung cancer cases are within 1-6 years of clinical diagnosis with cancer.

FIG. 15 depicts the cumulative incidence plot for lung cancer death amongst all asymptotic individuals who were test-positive or test-negative based on 4MP plus PLCOₘ₂₀₁₂ scores at a 1.7% 6-year risk threshold. Analyses were based on cases with ≥10 PYS smoking history and all lung cancer cases are within 1-6 years of clinical diagnosis with cancer.

FIG. 16 depicts the cumulative incidence plot for lung cancer death and non-lung cancer death diagnosed within 1-6 years of blood draw who were test-positive or test-negative based on 4MP plus PLCOₘ₂₀₁₂ scores at a 1.7% 6-year risk threshold. Analyses were based on cases with ≥10 PYS smoking history.

FIG. 17 depicts the cumulative incidence plot for lung cancer death and non-lung cancer death diagnosed within 1-6 years of blood draw who were test-positive or test-negative based on 4MP plus PLCOₘ₂₀₁₂ scores at a 1.0% 6-year risk threshold. Analyses were based on cases with ≥10 PYS smoking history.

### DETAILED DESCRIPTION

Provided herein is a method of method of predicting the prognosis of an individual having a lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
wherein the combined model score indicates the prognosis of the individual with respect to time to progression and overall survival.

In some embodiments, the individual is predicted as having a poor lung cancer survival prognosis when the combined model score is above a pre-defined risk threshold.

Also provided is a method of predicting disease outcome in an individual having lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having an increased risk of a poor disease outcome when the combined model score is above is above a pre-defined risk threshold.

Also provided is a method for identifying an individual at high risk of lung cancer death, comprising:
determining a combined model score using a logistic regression with a PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having a high risk of lung cancer death when the combined model score is above is above a pre-defined risk threshold.

In some embodiments, the PLCOₘ₂₀₁₂ risk model score is based on baseline questionnaire information comprising age, race or ethnic group, education, body mass index, chronic obstructive pulmonary disease, personal history of cancer, family history of lung cancer and smoking status (current v former), intensity, duration, and quit time.

In some embodiments, the combined model score is calculated with the equation: −11.836+1.6160*(0.4730*log[CA125]+0.6531*log[CEA]+0.2612*log[CYFRA21-1]+0.9238*log[Pro-SFTPB])+0.9861*(PLCOₘ₂₀₁₂ score).

In some embodiments, the pre-defined risk threshold is the 1.0% 6-year risk threshold.

In some embodiments, a combined model score greater than −4.595 is considered a positive test.

In some embodiments, the pre-defined risk threshold is the 1.7% 6-year risk threshold.

In some embodiments, a combined model score is greater than −4.057 is considered a positive test.

In some embodiments, the individual is asymptomatic.

In some embodiments, the individual is high-risk.

In some embodiments, a method described herein further comprises calculating the life span of the individual.

In some embodiments, the levels of biomarkers CEA, CA125, CYFRA21-1, and Pro-SFTPB in a biological sample obtained from the individual are determined by an immunoassay.

In some embodiments, each of the biomarkers CEA, CA125, CYFRA21-1, and Pro-SFTPB in a biological sample obtained from the individual generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the lung cancer is early stage (e.g., stage I or II).

In some embodiments, the lung cancer is advanced stage (e.g., stage III or IV).

In some embodiments, the individual has a smoking history of ≥10 pack years.

In some embodiments, the individual is between the age of 50 and 80 years.

In some embodiments, the AUC of the method is greater than the AUC for a different biomarker, biomarkers, panel, assay, algorithm, model, or any combination thereof.

In some embodiments, the AUC is greater than 0.85.

In some embodiments, the AUC is between 0.86 and 0.90.

In some embodiments, the AUC is about 0.88.

In some embodiments, the sensitivity and specificity values at a ≥1.7%/6-year risk threshold of the method are greater than the sensitivity and specificity values for a different biomarker, biomarkers, panel, assay, algorithm, model or any combination thereof.

In some embodiments, the sensitivity is greater than 0.80 and the specificity is greater than 0.65.

In some embodiments, the sensitivity is between 0.82 and 0.91 and the specificity is between 0.70 and 0.72.

In some embodiments, the sensitivity is about 0.85 and the specificity is about 0.71.

In some embodiments, the sensitivity and specificity values at a ≥1.0%/6-year risk threshold of the method are greater than the sensitivity and specificity values for a different biomarker, biomarkers, panel, assay, algorithm, model or any combination thereof.

In some embodiments, the sensitivity is greater than 0.85 and the specificity is greater than 0.55.

In some embodiments, the sensitivity is between 0.87 and 0.94 and the specificity is between 0.56 and 0.59.

In some embodiments, the sensitivity is about 0.90 and the specificity is about 0.58.

In some embodiments, the individual is subsequently designated for further lung cancer screening or treatment.

In some embodiments, the screening is chosen from endoscopic ultrasound, magnetic resonance imaging (MRI), and computed topography (CT) scans.

In some embodiments, the screening is performed annually.

In some embodiments, the screening is performed semi-annually.

In some embodiments, the treatment is chosen from surgery, chemotherapy, immunotherapy, radiation therapy, targeted therapy, or a combination thereof.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

As used herein, “lung cancer” refers to a malignant neoplasm of the lung characterized by the abnormal proliferation of cells, in which the growth of the cells exceeds and is uncoordinated with that of the normal tissues around it. In some embodiments, lung cancer may vary in severity, represented by stages I through IV. In some embodiments, lung cancer may be in an early stage (e.g., stage I or II), or it may be advanced (e.g., stage III or IV).

As used herein, the terms “subject” or “patient” refer to a mammal, preferably a human, for whom a classification as lung cancer-positive or lung cancer-negative is desired, and for whom further treatment can be provided.

As used herein, “healthy” refers to an individual in whom no evidence of lung cancer is found, i.e., the individual does not have lung cancer. Such an individual may be classified as “lung cancer-negative” or as having healthy lungs, or normal, non-compromised lung function. A healthy patient or subject has no symptoms of lung cancer, but may have benign lung nodules or masses, i.e., a combination of adenomas and cysts, or a non-cancerous lung condition or conditions, such as chronic obstructive pulmonary disease (COPD). In some embodiments, a healthy patient or subject may be used as a comparison to diseased or suspected diseased samples for determination of lung cancer in a patient or a group of patients.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery. Pharmacological substances as used herein may include, but are not limited to, anticancer drugs including chemotherapeutics, polyamine inhibitors, hormone therapies, and targeted therapies. Examples of chemotherapeutics for lung cancer include paclitaxel/Taxol (e.g. albumin bound paclitaxel or nab-paclitaxel, trade name Abraxane®), erlotinib (Tarceva® and others), afatinib (Gilotrif®), gefitinib (Iressa®), bevacizumab (Avastin®), gemcitabine (Gemzar®), crizotinib (Xalkori®), ceritinib (Zykadia®), cisplatin/Platinol, carboplatin (Paraplatin®), docetaxel (Taxotere®), pemetrexed (Alimta®), and vinorelbine (Navelbine®); as well as combination regimens of chemotherapy including cisplatin+paclitaxel, TIP (paclitaxel/Taxol, ifosfamide, and cisplatin/Platinol), VeIP (vinblastine, ifosfamide, and cisplatin/Platinol), VIP (etoposide/VP-16, ifosfamide, and cisplatin/Platinol), VAC (vincristine, dactinomycin, and cyclophosphamide), and PEB (cisplatin/Platinol, etoposide, and bleomycin). The terms “pharmacological substance” and “anticancer therapy” may also include substances used in immunotherapy, such as checkpoint inhibitors. Treatment may include a multiplicity of pharmacological substances, or a multiplicity of treatment methods, including, but not limited to, surgery and chemotherapy.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc.), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “elevated” refers to a biomarker level or model score in a given subject that is greater relative to the same biomarker level or model score in a given set of healthy patients or subjects. In some embodiments, an elevated PLCOₘ₂₀₁₂ model score is 0.00948 or greater. In some embodiments, an elevated PLCOₘ₂₀₁₂ model score is 0.016082 or greater.

As used herein, the term “hazard ratio” refers to a measure of how often a particular event happens in one group compared to how often it happens in another group, over time. Hazard ratios are often used in clinical trials to measure survival at any point in time in a group of patients who have been given a specific treatment compared to a control group given another treatment or a placebo. Hazard is defined as the slope of the survival curve—a measure of how rapidly subjects are dying. A hazard ratio of one means that there is no difference in survival between the two groups. A hazard ratio of greater than one or less than one means that survival was better in one of the groups. If the hazard ratio is 2.0, then the rate of deaths in one treatment group is twice the rate in the other group.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for lung cancer positive.

As used herein, the term “logistic regression” refers to a regression method in which the assignment of a prediction from the model can have one of several allowed discrete values. For example, the logistic regression models used herein can assign a prediction, for a certain subject, of either lung cancer-positive or lung cancer-negative.

As used herein, the term “biomarker score” refers to a numerical score for a given biomarker measured in a sample from a subject. The biomarker score is calculated by normalizing or weighting the measured level using a fixed coefficient as prescribed by the statistical method for a given biomarker panel. Biomarker scores are used as components in calculating a risk score for the subject. Elevated biomarker scores will carry more weight in risk score calculations and can indicate a higher risk for lung cancer for the subject.

As used herein, the term “risk score” refers to a single numerical value that indicates an asymptomatic human subject's risk for lung cancer as compared to the known prevalence of lung cancer in the disease cohort. The risk score is calculated through adding together the parameters of a statistical method derived from the subject for a given biomarker panel, which may take the form of biomarker scores, statistical model scores, or model constants. A higher risk score correlates to a higher risk for lung cancer in the subject. The risk score is empirically derived and will change depending on the data, cohort of the subject population, type of lung cancer, biomarkers chosen, occupational and environmental factors, and so on. In certain embodiments, the risk score as calculated for the human subject is the summation of the biomarker scores obtained from the subject. In certain embodiments, the risk score as calculated for the human subject is the summation of the biomarker scores obtained from the subject and one or more additional model constants. In certain embodiments, the risk score as calculated for a human subject is the summation of the biomarker scores obtained for the subject, normalized scores from one or more additional statistical models based on risk factors for the subject, and one or more additional model constants.

As used herein, the term “risk profile” refers to an assessment of a patient's risk score compared to those of a plurality of patients assessed using the same model, in which the patient is placed into an appropriate risk group based on a given score threshold. The score threshold is empirically derived and will change depending on the data, cohort of the subject population, type of lung cancer, biomarkers chosen, occupational and environmental factors, and so on. In certain embodiments, the patient's risk score exceeds the score threshold and their risk profile classifies them as being at risk for lung cancer (“positive”). In certain embodiments, the patient's risk profile is lower than the score threshold and classifies them as not being at risk for lung cancer (“negative”). In some embodiments, the score threshold is 0.005, or 0.5%, or greater. In some embodiments, the score threshold is 0.01, or 1%, or greater. In some embodiments, the score threshold is 0.05, or 5%, or greater. In some embodiments, the score threshold is 0.1, or 10%, or greater.

As used herein, the term “cutoff” or “cutoff point” refers to a mathematical value associated with a specific statistical method that can be used to assign a classification of lung cancer-positive of lung cancer-negative to a subject, based on said subject's biomarker score.

As used herein, when a numerical value above or below a cutoff value “is characteristic of lung cancer,” what is meant is that the subject, analysis of whose sample yielded the value, either has lung cancer or is at risk for lung cancer.

As used herein, the “use” of markers for diagnosing lung cancer refers to quantification of the levels or amounts in a biological sample of one or more markers described herein. Quantification may be done using any known methods or techniques in the art or described herein. In some embodiments, markers may be used or combined together as a panel for statistical comparison to other samples.

In some embodiments, using the markers pro-SFTPB, CA125, CEA, CYFRA21-1, and the PLCOₘ₂₀₁₂ model score together as a panel may have an AUC (95% CI) of 0.84 or greater, including about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, analyzing any of the marker panels described herein for diagnosis of lung cancer using fixed coefficients may result in an AUC (95% CI) of from about 0.55 to about 0.88 for distinguishing early-stage lung cancer, e.g., about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, or the like. In some embodiments, analyzing these marker panels using fixed coefficients may result in an AUC (95% CI) of 0.86 for distinguishing early-stage lung cancer.

As used herein, a subject who is at “risk for lung cancer” is one who may not yet evidence overt symptoms of lung cancer, but who is producing levels of biomarkers which indicate that the subject has lung cancer or may develop it in the near term. A subject who has lung cancer or is suspected of harboring lung cancer may be treated for the cancer or suspected cancer.

As used herein, the term “classification” refers to the assignment of a subject as being at risk for lung cancer or not being at risk for lung cancer, based on the result of the biomarker score, risk score, or risk profile that is obtained for said subject.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or the risk for lung cancer in subjects of a certain population.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, DAS, arginine, and creatine riboside.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for lung cancer-positive and lung cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “positive predictive value” refers to the proportion of positive results derived by a certain method that are truly positive.

As used herein, the term “disease progression” or “early disease progression” is defined as upgrading of Gleason score and/or increased tumor volume on surveillance biopsy within 18 months after start of active surveillance.

The phrase “therapeutically effective” is intended to qualify the amount of active ingredients used in the treatment of a disease or disorder or on the effecting of a clinical endpoint.

### List of Abbreviations

4MP=four-marker protein panel (pro-surfactant protein B (pro-SFTPB), Mucin 16 (CA125), carcinoembryonic antigen (CEA), and cytokeratin-19 fragment (CYFRA21-1)); AUC=area under the curve; NSCLC=Non-small-cell lung carcinoma; SCLC=Small-cell lung carcinoma; PPV=positive predictive value; ROC=receiver operating characteristic.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: Mortality Benefit of a Blood-Based Biomarker Panel for Lung Cancer Based on the PLCO Cohort

A study was performed to investigate the utility of integrating a panel of circulating protein biomarkers in combination with a risk model based on subject characteristics to identify individuals at high risk of harboring a lethal lung cancer. As described further below, data from an established logistic regression model that combines 4-marker protein panel (4MP) together with the PLCOₘ₂₀₁₂ risk model assayed in pre-diagnostic sera from 552 lung cancer cases and 2,193 non-cases from the Prostate Lung Colorectal and Ovarian (PLCO) cohort were used in the study. Of the 552 lung cancer cases, 387 (70%) died from lung cancer. The cumulative incidence of lung cancer death as well as sub-distributional and cause-specific hazard ratios were calculated based on 4MP+PLCOₘ₂₀₁₂ risk scores at a pre-defined 1.0% and 1.7% 6-year risk thresholds, which correspond to the former and current US Preventive Services Task Force screening criteria, respectively.

When considering cases diagnosed within 1 year of blood draw and all non-cases, the AUC estimate of the 4MP+PLCOₘ₂₀₁₂ model for risk prediction of lung cancer death was 0.88 (95% CI: 0.86-0.90). The cumulative incidence of lung cancer death was statistically significantly higher in individuals with 4MP+PLCOₘ₂₀₁₂ scores above the 1.0% 6-year risk threshold (modified χ2: 166.27, P<0.0001). Corresponding sub-distributional and lung cancer death-specific hazard ratios for test-positive cases were 9.88 (95% CI: 6.44-15.18) and 10.65 (95% CI: 6.93-16.37), respectively.

### The PLCO Cohort

The PLCO Cancer Screening Trial was a randomized multicenter trial in the United States which aimed at evaluating the impact of early detection procedures for prostate, lung, colorectal and ovarian cancer on disease-specific mortality. A biorepository was created for blood specimens that were annually collected from consented, intervention group participants. Reporting of cancer status was based on annual questionnaires. Medical records were obtained to document diagnostic follow-up and characteristics of any diagnosed lung cancers. The TNM stage and stage group were determined by the fifth edition of the American Joint Committee on Cancer's Cancer Staging Manual. Treatment data were abstracted from medical records for the 1-year period following diagnosis. PLCO participants were followed for an additional 13 years after the PLCO study ended for lung cancer incidence and 20 years for lung cancer death.

All deaths occurring during the trial were ascertained primarily through annual study update questionnaires. Participants who did not return the questionnaire were contacted by repeat mailing or telephone. To enhance the completeness of end point verification, the active follow-up was accompanied by periodic linkage to the National Death Index. Death certificates were obtained to confirm the death and to determine the provisional cause of death. As the underlying cause of death was not always accurately recorded on the death certificate, the PLCO trial used an end-point adjudication process to assign cause of death in a uniform and unbiased manner. All deaths with causes potentially related to cancer were reviewed by a death review committee with a nonvoting chair and three experience reviewers. Death reviewers were blinded to the trial group of the deceased participant. Lung cancer-specific deaths were defined as those with underlying cause of lung cancer or treatment for lung cancer.

### Risk Model Based on Subject Characteristics

PLCOₘ₂₀₁₂ is a survey-based logistic-regression model that predicts the six-year risk of lung cancer diagnosis. This duration was chosen to optimize application and testing in the National Lung Screening Trial (NLST), which had a median six years of follow-up. Predictive variables in the PLCOₘ₂₀₁₂ model were obtained through baseline questionnaire information, and include age, race/ethnic group, education, body mass index, chronic obstructive pulmonary disease, personal history of cancer, family history of lung cancer and smoking status (current vs. former), intensity, duration, and quit time. The details of the PLCOₘ₂₀₁₂ model and its implementation have been described in Tammemagi et al., 2013, NEJM 368:728-736, which is hereby incorporated by reference in its entirety.

### 4MP Readouts in the PLCO Specimen Set

The specimen set consisted of sera collected preceding a lung cancer diagnosis from 552 cases and 2,193 non-case PLCO participants that did not receive a lung cancer diagnosis during the study trial or within the 13-year study follow-up period. Biomarker scores for the 4MP were calculated based on a logistic regression model. The combined model of the 4MP+PLCOₘ₂₀₁₂ for predicting lung cancer within one year was developed by fitting a logistic regression with the 4MP score and the linear predictor of the PLCOₘ₂₀₁₂ as two separate predictors.

### Statistical Analyses

Predefined weights and cutpoints based on 1.0% and 1.7% 6-year risk thresholds for the 4MP score, PLCOₘ₂₀₁₂ score, and the 4MP+PLCOₘ₂₀₁₂ score were applied. Risk thresholds of ≥1.0% and ≥1.7% 6-year risk were used, which have been shown to result respectively in similar numbers of screening eligible individuals as the USPSTF2021 and USPSTF2013 screening criteria. Given the limited number of cases with <10 PY smoking history in the study specimen set, analyses were focused on those participants with ≥10 PY and stratified them into low-, medium-, and high-risk groups defined by pack-years and years since quitting (Tables 1-4).

TABLE 1
Specimen characteristics for participants diagnosed with lung cancer.

| | Death from | | Death From | | Alive at Last | |
|---|---|---|---|---|---|---|
| | Lung Cancer | | other Causes | | Follow-Up | |
| Time to | Within | From one to | Within | From one to | Within | From one to |
| Diagnosis | 1 year | six years | 1 year | six years | 1 year | six years |
| N | 250 | 644 | 66 | 186 | 33 | 62 |
| Stage, | | | | | | |
| N (%) | | | | | | |
| Stage I | 57 (23) | 156 (24) | 31 (47) | 88 (47) | 29 (88) | 49 (79) |
| Stage II | 15 (6) | 19 (3) | 2 (3) | 9 (5) | 2 (6) | 5 (8) |
| Stage III | 63 (25) | 164 (25) | 10 (15) | 40 (22) | 0 (0) | 0 (0) |
| Stage IV | 85 (34) | 249 (39) | 9 (14) | 32 (17) | 1 (3) | 0 (0) |
| Gender, | | | | | | |
| N (%) | | | | | | |
| Male | 168 (67) | 441 (68) | 47 (71) | 127 (68) | 14 (42) | 25 (40) |
| Female | 82 (33) | 203 (32) | 19 (29) | 96 (52) | 19 (58) | 37 (60) |
| Smokers, | | | | | | |
| N (%) | | | | | | |
| Current | 109 (44) | 294 (46) | 25 (38) | 76 (41) | 16 (48) | 28 (45) |
| Former | 141 (56) | 350 (54) | 41 (62) | 110 (59) | 17 (52) | 34 (55) |
| Pack-Year | | | | | | |
| <10 | 4† | 20 | 3† | 14 | 1 | 5 |
| ≥10 | 243 | 624 | 62 | 172 | 32 | 57 |

†4 cases did not have information regarding pack-year

TABLE 2
Specimen characteristics for non-case participants.

| | Death from | | Death From | | Alive at Last | |
|---|---|---|---|---|---|---|
| | Lung Cancer | | other Causes | | Follow-Up | |
| | | # of | | # of | | # of |
| | # of | unique | # of | unique | # of | unique |
| Total samples | specimens | patients | specimens | patients | specimens | patients |
| N | 27 | 8 | 1,847 | 526 | 6,456 | 1,556 |
| Gender, | | | | | | |
| N (%) | | | | | | |
| Male | 19 | 5 | 1,188 | 338 | 3,381 | 804 |
| Female | 8 | 3 | 659 | 188 | 3,075 | 752 |
| Smokers, | | | | | | |
| N (%) | | | | | | |
| Current | 19 | 6 | 441 | 128 | 1,006 | 248 |
| Former | 8 | 2 | 1,406 | 398 | 5,450 | 1,308 |
| Pack-Year | | | | | | |
| <10 | 0 | 0 | 210 | 61 | 1,311 | 314 |
| ≥10 | 27 | 8 | 1,637 | 465 | 5,145 | 1,242 |

TABLE 3
Cumulative 20-year incidence of lung cancer and lung cancer death
among different risk strata in the intervention arm of the PLCO.

| | | | PLCO intervention arm | |
|---|---|---|---|---|
| | | | 20-year cumulative | 20-year cumulative |
| Criteria | TF2013 | TF2021 | incidence of lung | incidence of lung cancer |
| <10PY | − | − | 0.0066 | 0.0053 |
| ≥10PY | NA | NA | 0.0612 | 0.0497 |
| 10 to <20PY | − | − | 0.0182 | 0.0150 |
| 20 to <30PY; ≥15QY | − | − | 0.0199 | 0.0184 |
| 20 to <30PY; <15QY | − | + | 0.0584 | 0.0497 |
| ≥30PY; ≥15QY | − | − | 0.0494 | 0.0385 |
| ≥30PY; <15QY | + | + | 0.0977 | 0.0790 |
| Total | | | 0.0520 | 0.0424 |

Abbreviations: PY, smoking pack years; QY, quit year.

TABLE 4
Survival time amongst lung-cancer cases diagnosed within one year of blood
draw stratified into different subgroups based on smoking history.

| | | Median survival time (years) for cases who |
|---|---|---|
| Smoking | Number at risk at | died from Lung Cancer |
| Strata† | baseline | (Median (1st-3rd quantile)) |
| All smokers | 387 | 2.77 (2.60-3.02) |
| >=10 PYs | 377 | 2.62 (2.48-2.84) |
| Low | 46 | Not reached |
| Med | 78 | 3.83 (3.37-5.20) |
| High | 272 | 2.36 (2.17-2.62) |

†Strata according to eligibility under USPSTF recommendations. Low: 10-20 smoking packs per year (PYs) or 20-29 smoking packs per year and smoking quit time ≥15 years; Medium- 20-29 smoking pack years and smoking quit time <15 years or 30+ smoking packs per years and smoking quit time >15 years; High- 30+ smoking packs per year and smoking quit time <15 years.

Strata (low-, medium-, and high-risk) specific cutpoints for the 4MP score (1.8206*4MP) were estimated. At the 1.0% 6-year risk threshold, respective 4MP scores greater than 13.579, 12.529, and 12.332 for the low, medium and high-risk strata were considered ‘test positive’. At the 1.7% 6-year risk threshold, respective 4MP scores greater than 14.117, 13.066, and 12.870 for low, medium and high-risk strata were considered ‘test positive’. For the combined 4MP+PLCOₘ₂₀₁₂, at the 1.0% and 1.7% 6-year risk thresholds, respective scores (−11.836+1.6160*4MP+0.9861*(PLCOₘ₂₀₁₂ score)) of greater than −4.595 and −4.057 were considered as ‘test positive’. For the PLCOₘ₂₀₁₂ score, the logit form of the PLCOₘ₂₀₁₂ risk model was used.

For AUC calculations, all cases specimens diagnosed within one year of blood draw and all non-cases specimens were considered. The event positive group was defined as those individuals who died from lung cancer, whereas the event negative group consisted of participants that did not die from lung cancer (which included censored information and other causes of death). Corresponding 95% confidence interval for statistical parameters were estimated using 1,000 bootstraps.

Survival analyses were performed among individuals with ≥10 pack-years of smoking history (PYs) to be consistent with prior studies. In the PLCO dataset, death due to causes other than lung cancer precludes the occurrence of lung cancer-specific mortality. In other words, an individual who dies from other non-lung cancer related causes is no longer at risk of lung cancer death. Therefore, alternative causes of death were considered as competing risk events. To estimate the incidence of lung cancer death over time in the presence of competing risks, two different modeling approaches were used: cause-specific hazard for lung cancer death (where non-lung cancer death is treated as a censoring event) and the sub-distributional hazard of the cumulative incidence function for lung cancer death.

For the cause-specific hazard function, the instantaneous hazard function of the k^{th} event (k denotes for lung cancer death or non-lung cancer death) is defined as:

$${λ}_{k}^{cs} = {lim}_{Δ t \to 0} \frac{Pr ( t \leq T < t + Δ t , D = k \vert T \geq t )}{Δ t}$$

For sub-distributional hazard ratios, the following modeling approach was followed. The sub-distributional hazard function focuses on risk of failure from the k^{th} event (k denotes lung cancer death or non-lung cancer death) in subjects who have not yet experienced an event of type k. This is defined as:

$${λ}_{k}^{sd} = {lim}_{Δ t \to 0} \frac{Pr ( t \leq T < t + Δ t , D = k \vert T > t ⋃ ( T < t ⋂ K \neq k ) )}{Δ t}$$

Time to event was defined as the time interval between blood draw until lung cancer death, other cause of death, or last period of follow-up. Two curves in the cumulative incidence plot were compared using Gray's modified χ² test statistics.

All analyses were performed in R (version 4.2.0) using the “pROC” package for calculating area under receiver operation characteristics curve (AUC), sensitivity and specificity metrics and the “cmprisk” package for time-dependent survival analyses.

### Predictive Performance of the Combined 4MP+PLCOₘ₂₀₁₂ Model for Lung Cancer-Specific Mortality

Of the 552 lung cancer cases diagnosed during the 6-year PLCO study period, 387 (70%) died from lung cancer, 99 (18%) died from other causes, 41(7%) were still alive at the time of last follow-up, and 25 (5%) did not have survival information available (Tables 1 and 5). Of the 2,193 non-case participants, 556 (25%) died from other causes (Table 2). Notably, 8 (0.004%) died from lung cancer after the 13-year follow-up period for lung cancer incidence. These 8 individuals were excluded from subsequent analyses.

TABLE 5
Patient characteristics and mortality outcomes for PLCO specimen set.

| | Lung Cancer Cases † | | | Non-Cases ‡ | |
|---|---|---|---|---|---|
| | Death from | Death from | Not Dead or | Death from | Not Dead or |
| | Lung Cancer | Other Causes | Missing | Other Causes | Missing |
| N, cases | 387 | 99 | 66 | 556 | 1,629 |
| Gender, N (%) | | | | | |
| Male | 256 (66.1) | 68 (68.7) | 30 (45.5) | 360 (64.7) | 845 (51.9) |
| Female | 131 (33.9) | 31 (31.3) | 36 (54.5) | 196 (35.3) | 784 (48.1) |
| Age, (median, IQR) | 66.0 (62.0-69.0) | 66 (63.0-70.0) | 60.0 (58.0-63.0) | 65 (61.0-70.0) | 60.0 (57.0-64.0) |
| Smokers, N (%) | | | | | |
| Current | 173 (44.7) | 39 (39.4) | 26 (39.4) | 134 (24.1) | 256 (15.7) |
| Former | 214 (55.3) | 60 (60.6) | 40 (60.6) | 422 (75.9) | 1,373 (84.3) |
| Smoking Pack Years | 51.0 (39.2-75.8) | 52.8 (35.0-78.0) | 45.5 (28.0-66.0) | 40 (18.5-58.0) | 24 (12.0-41.2) |
| (median, IQR) | | | | | |
| Pack-Years, N(%) | | | | | |
| <10 | 5 (1.3) | 4 (4) | 3 (4.5) | 63 (11.3) | 323 (19.8) |
| ≥10 | 377 (97.4) | 92 (92.9) | 63 (95.5) | 478 (86) | 1,265 (77.7) |
| Unknown | 5 (1.3) | 3 (3) | 0 (0) | 15 (2.7) | 41 (2.5) |
| Stage, N (%) | | | | | |
| Early (Stage I and II) | 106 (27.4) | 48 (48.5) | 49 (74.2) | — | — |
| Late (Stage III and IV) | 237 (61.2) | 34 (34.3) | 2 (3) | — | — |
| Unknown | 44 (11.4) | 17 (17.2) | 15 (22.7) | — | — |
| Subtype, N (%) | | | | | |
| NSCLC | 318 (82.2) | 88 (88.9) | 64 (97.0) | — | — |
| SCLC | 69 (17.8) | 11 (11.1) | 2 (3.0) | — | — |

Abbreviations: NSCLC: Non-small cell lung cancer; SCLC: Small cell lung cancer; IQR: interquartile range.
† Lung cancer cases diagnosed within the 6-year PLCO study period.
‡ 8 non-case participants were excluded as they developed lung cancer after the 13-year PLCO study follow-up period for lung cancer incidence

Median survival time for lung cancer cases diagnosed within one year of blood draw who died from lung cancer was 2.77 years (Interquartile range (IQR): 2.60-3.02 years) (Table 4).

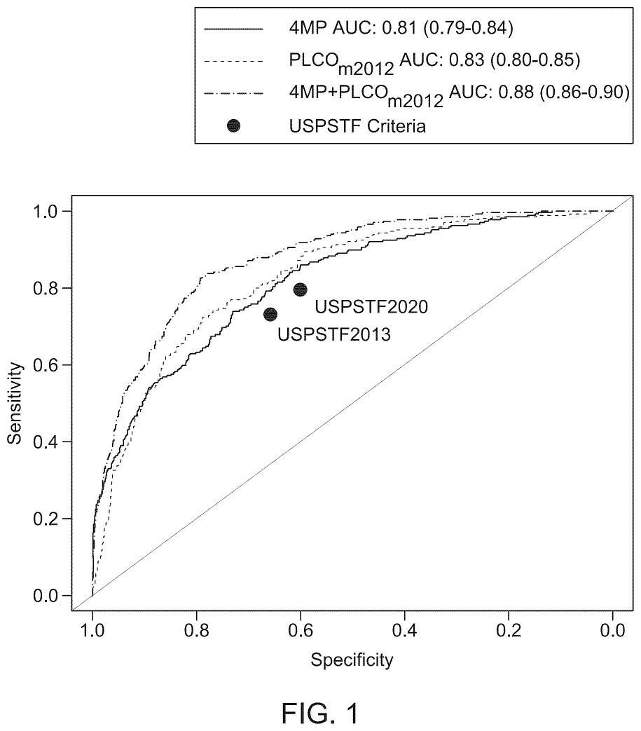

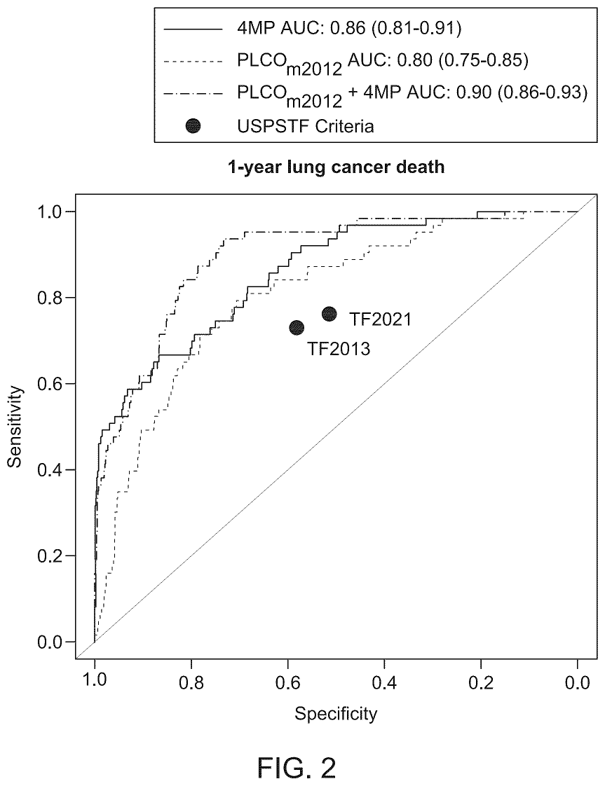

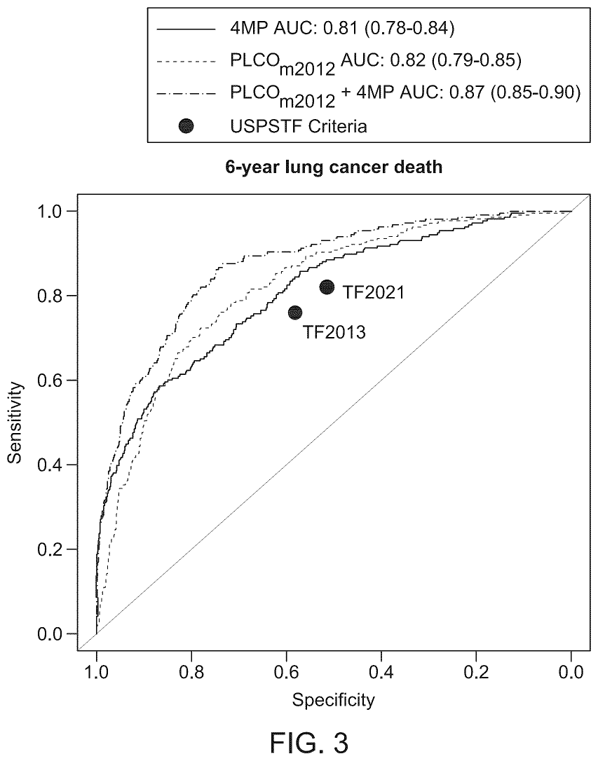

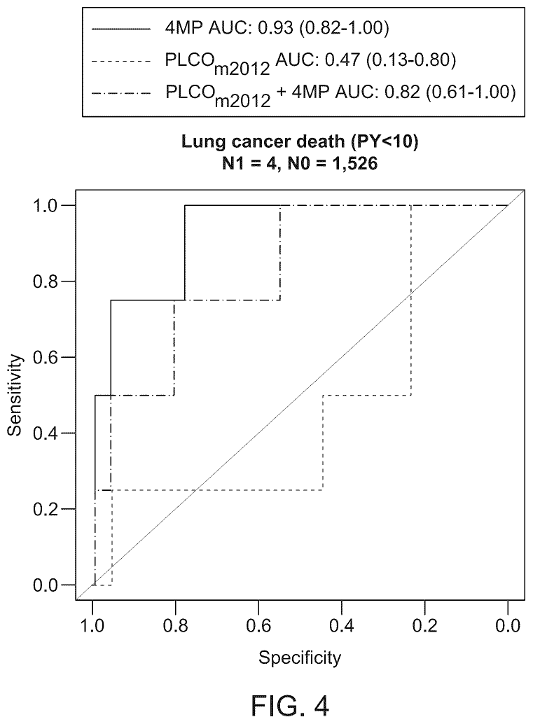

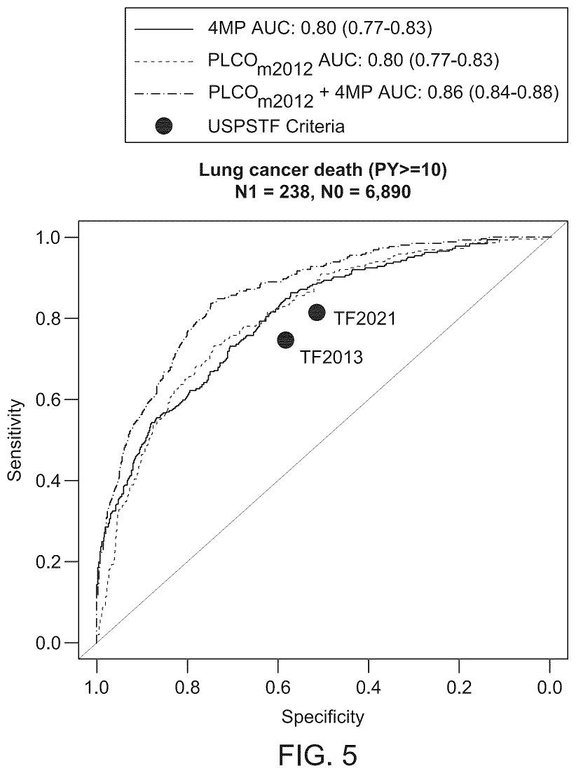

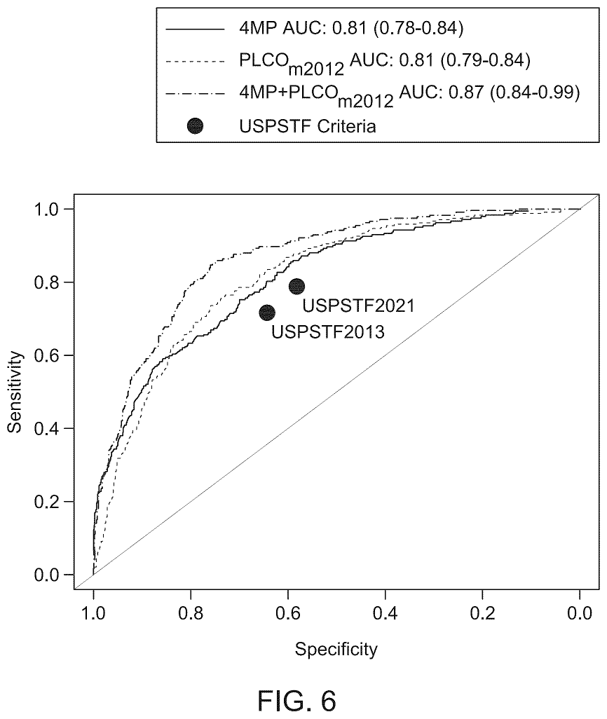

When considering sera collected within one year preceding a lung cancer diagnosis and all non-case sera, the combined 4MP+PLCOₘ₂₀₁₂ model had an AUC of 0.88 (95% CI: 0.86-0.90) for risk prediction of lung cancer specific mortality (FIGS. 1-5, Table 6). Similar performance estimates were found when considering unique randomly selected case and non-case sera (FIG. 6 and Table 7). Performance estimates of the combined 4MP+PLCOₘ₂₀₁₂ model for lung cancer-specific mortality from a non-small cell lung (NSCLC) or small cell lung cancer (SCLC) diagnosis were 0.87 (95% CI: 0.85-0.89) and 0.86 (95% CI: 0.82-0.90), respectively. Notably, when stratifying individuals into those with chronic obstructive pulmonary disease (COPD) and those without COPD, the 4MP+PLCOₘ₂₀₁₂ model yielded respective AUCs of 0.76 (95% CI: 0.69-0.84) and 0.88 (95% CI: 0.86-0.90) for predicting death due to lung cancer (Tables 6 and 7).

TABLE 6
Performance estimates of 4MP, PLCOₘ₂₀₁₂, and 4MP + PLCOₘ₂₀₁₂ for predicting death from
lung cancer. Case sera collected within 1 year of diagnosis and all non-case sera were considered.

| | | 4MP Only‡ | | | PLCOₘ₂₀₁₂‡ | | | 4MP + PLCOₘ₂₀₁₂‡ | | |
|---|---|---|---|---|---|---|---|---|---|---|
| | | | Sensi- | Speci- | | Sensi- | Speci- | | Sensi- | Speci- |
| | | | tivity | ficity | | tivity | ficity | | tivity | ficity |
| # lung | # other | | at 95% | at 95% | | at 95% | at 95% | | at 95% | at 95% |
| cancer | causes or | AUC | speci- | sensi- | AUC | speci- | sensi- | AUC | speci- | sensi- |
| deaths | no death | (95% CI) | ficity | tivity | (95% CI) | ficity | tivity | (95% CI) | ficity | tivity |

All

| | | | | | | | | | | |
|---|---|---|---|---|---|---|---|---|---|---|
| 242 | 8,416 | 0.82 | 0.38 | 0.34 | 0.83 | 0.34 | 0.42 | 0.88 | 0.47 | 0.51 |
| | | (0.79-0.84) | (0.31-0.43) | (0.25-0.45) | (0.80-0.85) | (0.29-0.40) | (0.32-0.51) | (0.86-0.90) | (0.41-0.53) | (0.46-0.61) |

Adenocarcinoma

| | | | | | | | | | | |
|---|---|---|---|---|---|---|---|---|---|---|
| 128 | 8,348 | 0.82 | 0.40 | 0.37 | 0.79 | 0.29 | 0.32 | 0.85 | 0.43 | 0.43 |
| | | (0.79-0.86) | (0.31-0.48) | (0.21-0.58) | (0.75-0.83) | (0.22-0.36) | (0.08-0.48) | (0.82-0.88) | (0.36-0.52) | (0.27-0.54) |

Squamous Cell Carcinoma

| | | | | | | | | | | |
|---|---|---|---|---|---|---|---|---|---|---|
| 78 | 8,327 | 0.78 | 0.23 | 0.36 | 0.81 | 0.34 | 0.34 | 0.86 | 0.33 | 0.56 |
| | | (0.73-0.82) | (0.13-0.32) | (0.24-0.48) | (0.77-0.86) | (0.24-0.45) | (0.32-0.51) | (0.83-0.89) | (0.22-0.44) | (0.49-0.64) |

NSCLC

| | | | | | | | | | | |
|---|---|---|---|---|---|---|---|---|---|---|
| 228 | 8,312 | 0.82 | 0.37 | 0.36 | 0.82 | 0.31 | 0.35 | 0.87 | 0.44 | 0.51 |
| | | (0.79-0.85) | (0.31-0.42) | (0.24-0.47) | (0.79-0.84) | (0.23-0.37) | (0.28-0.48) | (0.85-0.89) | (0.38-0.50) | (0.43-0.58) |

SCLC

| | | | | | | | | | | |
|---|---|---|---|---|---|---|---|---|---|---|
| 68 | 8,407 | 0.77 | 0.30 | 0.36 | 0.83 | 0.47 | 0.57 | 0.86 | 0.43 | 0.50 |
| | | (0.72-0.83) | (0.19-0.43) | (0.28-0.48) | (0.78-0.87) | (0.32-0.57) | (0.32-0.61) | (0.82-0.90) | (0.29-0.56) | (0.48-0.64) |

Stage I-II

| | | | | | | | | | | |
|---|---|---|---|---|---|---|---|---|---|---|
| 96 | 8,372 | 0.77 | 0.22 | 0.32 | 0.79 | 0.31 | 0.32 | 0.83 | 0.33 | 0.45 |
| | | (0.72-0.81) | (0.15-0.29) | (0.15-0.48) | (0.75-0.84) | (0.22-0.40) | (0.08-0.52) | (0.79-0.87) | (0.25-0.45) | (0.25-0.52) |

Stage III-IV

| | | | | | | | | | | |
|---|---|---|---|---|---|---|---|---|---|---|
| 171 | 8,323 | 0.83 | 0.42 | 0.38 | 0.83 | 0.38 | 0.43 | 0.89 | 0.50 | 0.56 |
| | | (0.81-0.86) | (0.35-0.51) | (0.31-0.54) | (0.80-0.86) | (0.30-0.46) | (0.32-0.57) | (0.87-0.91) | (0.41-0.57) | (0.49-0.66) |

Low-Risk Strata†

| | | | | | | | | | | |
|---|---|---|---|---|---|---|---|---|---|---|
| 19 | 2,417 | 0.73 | 0.42 | 0.15 | 0.67 | 0.16 | 0.05 | 0.79 | 0.42 | 0.32 |
| | | (0.59-0.88) | (0.21-0.63) | (0.13-0.26) | (0.55-0.80) | (0.05-0.37) | (0.04-0.48) | (0.69-0.89) | (0.21-0.63) | (0.29-0.63) |

Median-Risk Strata†

| | | | | | | | | | | |
|---|---|---|---|---|---|---|---|---|---|---|
| 48 | 1,599 | 0.74 | 0.19 | 0.18 | 0.73 | 0.17 | 0.24 | 0.78 | 0.31 | 0.39 |
| | | (0.67-0.80) | (0.08-0.31) | (0.11-0.57) | (0.66-0.80) | (0.06-0.25) | (0.05-0.47) | (0.72-0.84) | (0.15-0.44) | (0.18-0.51) |

High-Risk Strata†

| | | | | | | | | | | |
|---|---|---|---|---|---|---|---|---|---|---|
| 198 | 2,874 | 0.78 | 0.34 | 0.29 | 0.75 | 0.16 | 0.20 | 0.83 | 0.38 | 0.49 |
| | | (0.74-0.81) | (0.28-0.4) | (0.22-0.40) | (0.71-0.78) | (0.10-0.23) | (0.15-0.30) | (0.80-0.86) | (0.31-0.45) | (0.35-0.54) |

COPD

| | | | | | | | | | | |
|---|---|---|---|---|---|---|---|---|---|---|
| 37 | 440 | 0.72 | 0.24 | 0.18 | 0.72 | 0.16 | 0.29 | 0.76 | 0.27 | 0.35 |
| | | (0.63-0.81) | (0.11-0.38) | (0.08-0.35) | (0.64-0.79) | (0.05-0.30) | (0.20-0.44) | (0.69-0.84) | (0.11-0.43) | (0.19-0.53) |

Non-COPD

| | | | | | | | | | | |
|---|---|---|---|---|---|---|---|---|---|---|
| 232 | 7,976 | 0.82 | 0.39 | 0.36 | 0.83 | 0.35 | 0.36 | 0.88 | 0.48 | 0.51 |
| | | (0.80-0.85) | (0.33-0.47) | (0.26-0.49) | (0.80-0.85) | (0.29-0.42) | (0.29-0.50) | (0.86-0.90) | (0.41-0.53) | (0.45-0.59) |

TABLE 7
Performance estimates of 4MP, PLCOₘ₂₀₁₂, and 4MP + PLCOₘ₂₀₁₂ for predicting death from lung
cancer using single specimens from PLCO participants. Unique randomly selected case sera collected
within 1 year of diagnosis and unique randomly selected non-case sera were considered.

| | | 4MP Only‡ | | | PLCOₘ₂₀₁₂‡ | | | 4MP + PLCOₘ₂₀₁₂‡ | | |
|---|---|---|---|---|---|---|---|---|---|---|
| | | | Sensi- | Speci- | | Sensi- | Speci- | | Sensi- | Speci- |
| | | | tivity | ficity | | tivity | ficity | | tivity | ficity |
| # lung | # other | | at 95% | at 95% | | at 95% | at 95% | | at 95% | at 95% |
| cancer | causes or | AUC | speci- | sensi- | AUC | speci- | sensi- | AUC | speci- | sensi- |
| deaths | no death | (95% CI) | ficity | tivity | (95% CI) | ficity | tivity | (95% CI) | ficity | tivity |

All

| | | | | | | | | | | |
|---|---|---|---|---|---|---|---|---|---|---|
| 242 | 2,192 | 0.82 | 0.38 | 0.34 | 0.83 | 0.34 | 0.42 | 0.88 | 0.47 | 0.51 |
| | | (0.79-0.84) | (0.31-0.43) | (0.25-0.45) | (0.80-0.85) | (0.29-0.40) | (0.32-0.51) | (0.86-0.90) | (0.41-0.53) | (0.46-0.61) |

Adenocarcinoma

| | | | | | | | | | | |
|---|---|---|---|---|---|---|---|---|---|---|
| 106 | 2,125 | 0.82 | 0.40 | 0.37 | 0.79 | 0.29 | 0.32 | 0.85 | 0.43 | 0.43 |
| | | (0.79-0.86) | (0.31-0.48) | (0.21-0.58) | (0.75-0.83) | (0.22-0.36) | (0.08-0.48) | (0.82-0.88) | (0.36-0.52) | (0.27-0.54) |

Squamous Cell Carcinoma

| | | | | | | | | | | |
|---|---|---|---|---|---|---|---|---|---|---|
| 56 | 2,106 | 0.78 | 0.23 | 0.36 | 0.81 | 0.34 | 0.34 | 0.86 | 0.33 | 0.56 |
| | | (0.73-0.82) | (0.13-0.32) | (0.24-0.48) | (0.77-0.86) | (0.24-0.45) | (0.32-0.51) | (0.83-0.89) | (0.22-0.44) | (0.49-0.64) |

NSCLC

| | | | | | | | | | | |
|---|---|---|---|---|---|---|---|---|---|---|
| 202 | 2,183 | 0.82 | 0.37 | 0.36 | 0.82 | 0.31 | 0.35 | 0.87 | 0.44 | 0.51 |
| | | (0.79-0.85) | (0.31-0.42) | (0.24-0.47) | (0.79-0.84) | (0.23-0.37) | (0.28-0.48) | (0.85-0.89) | (0.38-0.50) | (0.43-0.58) |

SCLC

| | | | | | | | | | | |
|---|---|---|---|---|---|---|---|---|---|---|
| 48 | 2,091 | 0.77 | 0.30 | 0.36 | 0.83 | 0.47 | 0.57 | 0.86 | 0.43 | 0.50 |
| | | (0.72-0.83) | (0.19-0.43) | (0.28-0.48) | (0.78-0.87) | (0.32-0.57) | (0.32-0.61) | (0.82-0.90) | (0.29-0.56) | (0.48-0.64) |

Stage 1-11

| | | | | | | | | | | |
|---|---|---|---|---|---|---|---|---|---|---|
| 75 | 2,150 | 0.77 | 0.22 | 0.32 | 0.79 | 0.31 | 0.32 | 0.83 | 0.33 | 0.45 |
| | | (0.72-0.81) | (0.15-0.29) | (0.15-0.48) | (0.75-0.84) | (0.22-0.40) | (0.08-0.52) | (0.79-0.87) | (0.25-0.45) | (0.25-0.52) |

Stage III-IV

| | | | | | | | | | | |
|---|---|---|---|---|---|---|---|---|---|---|
| 147 | 2,102 | 0.83 | 0.42 | 0.38 | 0.83 | 0.38 | 0.43 | 0.89 | 0.50 | 0.56 |
| | | (0.81-0.86) | (0.35-0.51) | (0.31-0.54) | (0.80-0.86) | (0.30-0.46) | (0.32-0.57) | (0.87-0.91) | (0.41-0.57) | (0.49-0.66) |

Low-Risk Strata†

| | | | | | | | | | | |
|---|---|---|---|---|---|---|---|---|---|---|
| 17 | 609 | 0.73 | 0.42 | 0.15 | 0.67 | 0.16 | 0.05 | 0.79 | 0.42 | 0.32 |
| | | (0.59-0.88) | (0.21-0.63) | (0.13-0.26) | (0.55-0.80) | (0.05-0.37) | (0.04-0.48) | (0.69-0.89) | (0.21-0.63) | (0.29-0.63) |

Median-Risk Strata†

| | | | | | | | | | | |
|---|---|---|---|---|---|---|---|---|---|---|
| 42 | 414 | 0.74 | 0.19 | 0.18 | 0.73 | 0.17 | 0.24 | 0.78 | 0.31 | 0.39 |
| | | (0.67-0.80) | (0.08-0.31) | (0.11-0.57) | (0.66-0.80) | (0.06-0.25) | (0.05-0.47) | (0.72-0.84) | (0.15-0.44) | (0.18-0.51) |

High-Risk Strata†

| | | | | | | | | | | |
|---|---|---|---|---|---|---|---|---|---|---|
| 179 | 789 | 0.78 | 0.34 | 0.2 | 0.75 | 0.16 | 0.20 | 0.83 | 0.38 | 0.49 |
| | | (0.74-0.81) | (0.28-0.40) | (0.22-0.40) | (0.71-0.78) | (0.10-0.23) | (0.15-0.30) | (0.80-0.86) | (0.31-0.45) | (0.35-0.54) |

COPD

| | | | | | | | | | | |
|---|---|---|---|---|---|---|---|---|---|---|
| 33 | 127 | 0.72 | 0.24 | 0.18 | 0.72 | 0.16 | 0.29 | 0.76 | 0.27 | 0.35 |
| | | (0.63-0.81) | (0.11-0.38) | (0.08-0.35) | (0.64-0.79) | (0.05-0.30) | (0.20-0.44) | (0.69-0.84) | (0.11-0.43) | (0.19-0.53) |

Non-COPD

| | | | | | | | | | | |
|---|---|---|---|---|---|---|---|---|---|---|
| 209 | 2,065 | 0.82 | 0.39 | 0.36 | 0.83 | 0.35 | 0.36 | 0.88 | 0.48 | 0.51 |
| | | (0.80-0.85) | (0.33-0.47) | (0.26-0.49) | (0.80-0.85) | (0.29-0.42) | (0.29-0.50) | (0.86-0.90) | (0.41-0.53) | (0.45-0.59) |

### Comparison of the 4MP+PLCOₘ₂₀₁₂ Model Versus USPSTF Criteria for Predicting Lung-Cancer Specific Mortality Who Smoked≥10 PYs

The sensitivity and specificity of the combined 4MP+PLCOₘ₂₀₁₂ model were compared next to that of the USPSTF2013 and USPSTF2021 criteria for predicting lung cancer specific mortality. In comparison to USPSTF2013 criteria, corresponding to ≥1.7% 6-year risk threshold, the combined 4MP+PLCOₘ₂₀₁₂ model had improved sensitivity (85.0 (95% CI: 81.8-90.7) versus 74.0 (95% CI: 68.0-79.0)), specificity (71.0 (95% CI: 70.1-72.2) versus 58.0 (95% CI:57.0-59.0)), and positive predictive value (PPV) (24.2% (95% CI: 22.8-25.1) versus 16.3% (95% CI: 15.1-17.9)) for predicting lung cancer death. At the ≥1.0% 6-year risk threshold, corresponding to the USPSTF2021 criteria, the combined 4MP+PLCOₘ₂₀₁₂ model exhibited overall improved sensitivity of 90.2% (95% CI: 87.1%-94.2%) versus 81.0 (95% CI: 75.7-85.0), specificity of 58.1 (95% CI: 56.0-59.1) versus 52.0 (95% CI: 50.0-53.0), and PPV of 19.3% (95% CI: 18.1-20.4) versus 16.0% (95% CI: 13.9-17.4) for predicting lung cancer-specific mortality (Tables 8-10).

TABLE 8
Performance estimates of 4MP, PLCOₘ₂₀₁₂, and 4MP + PLCOₘ₂₀₁₂ for predicting death from lung cancer at 1.7% and 1.0% 6-year
risk thresholds. Case sera collected within 1 year of diagnosis and all non-case sera who smoked ≥10 PYs were considered.
Sensitivity and specificity based on USPSTF2013 and 2021 criteria corresponding to 1.7% and 1.0% 6-year risk thresholds are provided.

| | # lung | # other | 1.7% 6-year risk | | | | 1.0% 6-year risk | | |
|---|---|---|---|---|---|---|---|---|---|
| | cancer | causes or | Specificity | Sensitivity | PPV* | | Specificity | Sensitivity | PPV |
| Criteria | deaths | no death | (95% CI) | (95% CI) | (95% CI) | Criteria | (95% CI) | (95% CI) | (95% CI) |
| USPSTF2013 | 238 | 6,890 | 0.58 | 0.74 | 0.16 | USPSTF2021 | 0.52 | 0.81 | 0.16 |
| | | | (0.57-0.59) | (0.68-0.79) | (0.15-0.18) | | (0.50-0.53) | (0.76-0.85) | (0.14-0.17) |
| PLCOₘ₂₀₁₂ | | | 0.67 | 0.78 | 0.20 | PLCOₘ₂₀₁₂ | 0.48 | 0.91 | 0.16 |
| | | | (0.66-0.68) | (0.73-0.83) | (0.19-0.22) | | (0.47-0.49) | (0.88-0.94) | (0.15-0.17) |
| 4MP | | | 0.64 | 0.86 | 0.21 | 4MP | 0.47 | 0.94 | 0.16 |
| | | | (0.63-0.65) | (0.81-0.90) | (0.20-0.22) | | (0.46-0.48) | (0.91-0.97) | (0.15-0.17) |
| 4MP + | | | 0.71 | 0.85 | 0.24 | 4MP + | 0.58 | 0.90 | 0.19 |
| PLCOₘ₂₀₁₂ | | | (0.70-0.72) | (0.81-0.90) | (0.23-0.25) | PLCOₘ₂₀₁₂ | (0.56-0.59) | (0.87-0.94) | (0.18-0.20) |

*PPV were estimated by the classical Bayesian formula and the prevalence of the lung cancer death were estimated from intervention arm of the PLCO

TABLE 9
Performance estimates of 4MP, PLCOₘ₂₀₁₂, and 4MP + PLCOₘ₂₀₁₂ for predicting 1-year lung cancer death at 1.7% and 1.0% 6-year
risk thresholds. Case sera collected within 1 year of diagnosis and all non-case sera who smoked ≥10 PYs were considered. Sensitivity
and specificity based on USPSTF2013 and 2021 criteria corresponding to 1.7% and 1.0% 6-year risk thresholds are provided.

| | # lung | # other | 1.7% 6-year risk | | | | 1.0% 6-year risk | | |
|---|---|---|---|---|---|---|---|---|---|
| | cancer | causes or | Specificity | Sensitivity | PPV* | | Specificity | Sensitivity | PPV |
| Criteria | deaths | no death | (95% CI) | (95% CI) | (95% CI) | Criteria | (95% CI) | (95% CI) | (95% CI) |
| USPSTF2013 | 63 | 6,890 | 0.58 | 0.73 | 0.16 | USPSTF2021 | 0.52 | 0.77 | 0.15 |
| | | | (0.57-0.59) | (0.611-0.84) | (0.14-0.18) | | (0.50-0.53) | (0.66-0.86) | (0.13-0.16) |
| PLCOₘ₂₀₁₂ | | | 0.67 | 0.81 | 0.21 | PLCOₘ₂₀₁₂ | 0.48 | 0.89 | 0.16 |
| | | | (0.66-0.68) | (0.722-0.9) | (0.19-0.23) | | (0.47-0.49) | (0.806-0.96) | (0.15-0.17) |
| 4MP | | | 0.64 | 0.92 | 0.22 | 4MP | 0.47 | 0.97 | 0.17 |
| | | | (0.63-0.65) | (0.844-0.98) | (0.21-0.23) | | (0.46-0.48) | (0.918-1) | (0.16-0.17) |
| 4MP + | | | 0.71 | 0.94 | 0.26 | 4MP + | 0.58 | 0.95 | 0.20 |
| PLCOₘ₂₀₁₂ | | | (0.70-0.72) | (0.87-0.99) | (0.24-0.27) | PLCOₘ₂₀₁₂ | (0.56-0.59) | (0.89-1) | (0.19-0.21) |

*PPV were estimated by the classical Bayesian formula and the prevalence of the lung cancer death were estimated from intervention arm of the PLCO

TABLE 10
Performance estimates of 4MP, PLCOₘ₂₀₁₂, and 4MP + PLCOₘ₂₀₁₂ for predicting 6-year lung cancer death at 1.7% and 1.0% 6-year
risk thresholds. Case sera collected within 1 year of diagnosis and all non-case sera who smoked ≥10 PYs were considered. Sensitivity
and specificity based on USPSTF2013 and 2021 criteria corresponding to 1.7% and 1.0% 6-year risk thresholds are provided.

| | # lung | # other | 1.7% 6-year risk | | | | 1.0% 6-year risk | | |
|---|---|---|---|---|---|---|---|---|---|
| | cancer | causes or | Specificity | Sensitivity | PPV* | | Specificity | Sensitivity | PPV |
| Criteria | deaths | no death | (95% CI) | (95% CI) | (95% CI) | Criteria | (95% CI) | (95% CI) | (95% CI) |
| USPSTF2013 | 218 | 6,890 | 0.58 | 0.82 | 0.17 | USPSTF2021 | 0.52 | 0.82 | 0.16 |
| | | | (0.57-0.59) | (0.76-0.86) | (0.16-0.18) | | (0.50-0.53) | (0.77-0.87) | (0.15-0.16) |
| PLCOₘ₂₀₁₂ | | | 0.67 | 0.87 | 0.21 | PLCOₘ₂₀₁₂ | 0.48 | 0.91 | 0.16 |
| | | | (0.66-0.68) | (0.82-0.91) | (0.2-0.22) | | (0.47-0.49) | (0.87-0.95) | (0.15-0.17) |
| 4MP | | | 0.64 | 0.86 | 0.21 | 4MP | 0.47 | 0.94 | 0.16 |
| | | | (0.63-0.65) | (0.81-0.90) | (0.20-0.22) | | (0.46-0.48) | (0.90-0.97) | (0.15-0.17) |
| 4MP + | | | 0.71 | 0.88 | 0.25 | 4MP + | 0.58 | 0.90 | 0.19 |
| PLCOₘ₂₀₁₂ | | | (0.70-0.72) | (0.83-0.92) | (0.24-0.26) | PLCOₘ₂₀₁₂ | (0.56-0.59) | (0.86-0.94) | (0.18-0.20) |

*PPV were estimated by the classical Bayesian formula and the prevalence of the lung cancer death were estimated from intervention arm of the PLCO

Relationship of 4MP+PLCOₘ₂₀₁₂ at 1.7% and 1.0% 6-Year Risk Thresholds with Incidence of Lung Cancer Death Among Individuals Who Smoked≥10 PYs.

Further lung cancer-specific survival analyses were performed. For these analyses, all cases where specimens were diagnosed within one year of blood draw were considered. All non-case individuals with ≥10 PY smoking history were dichotomized into test-positive or test-negative based on 4MP+PLCOₘ₂₀₁₂ model scores ≥ or <the 1.7% or 1.0% 6-year risk thresholds, respectively.

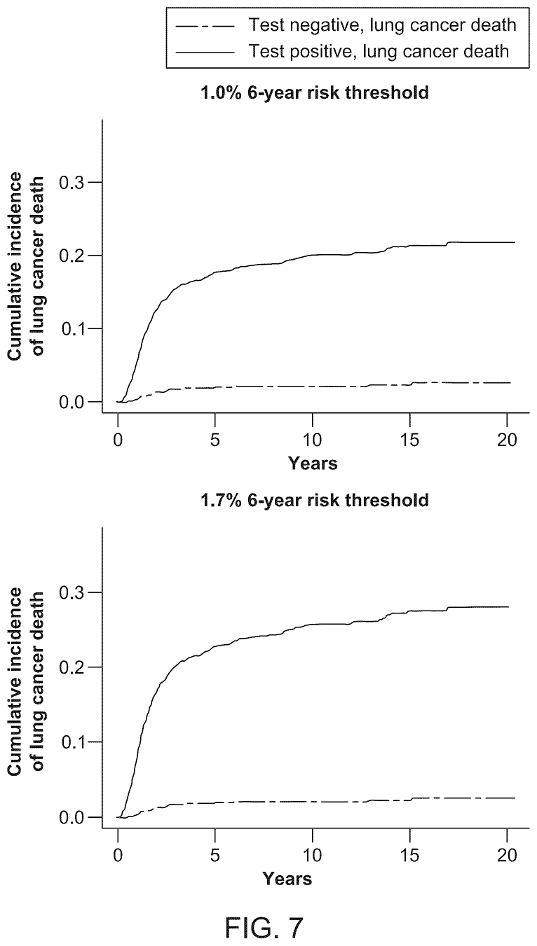

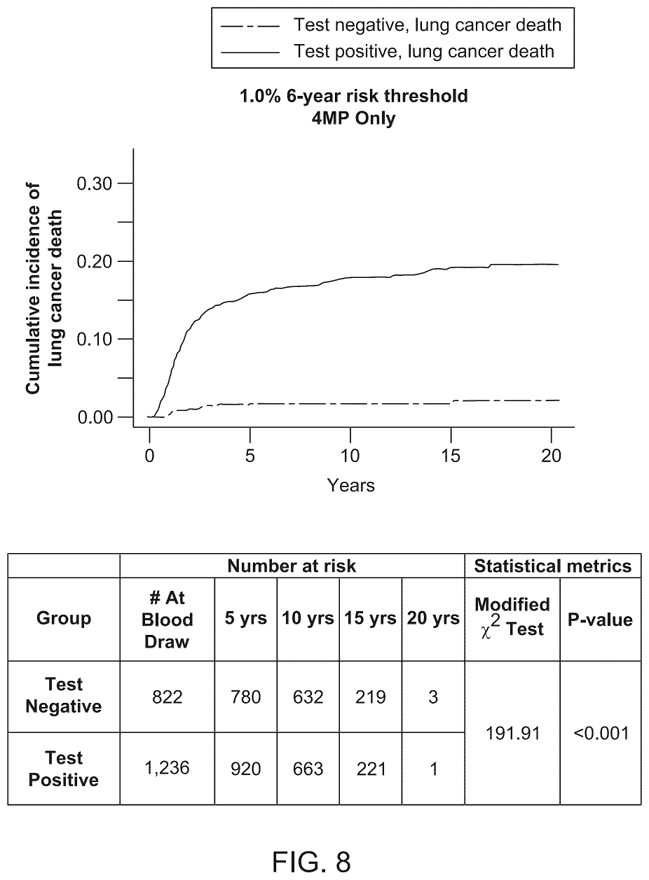

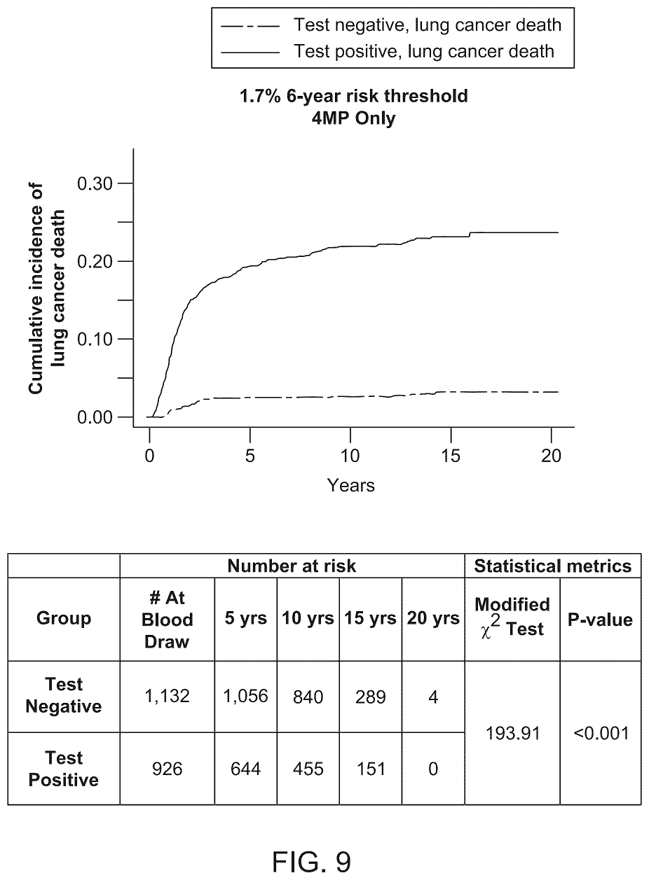

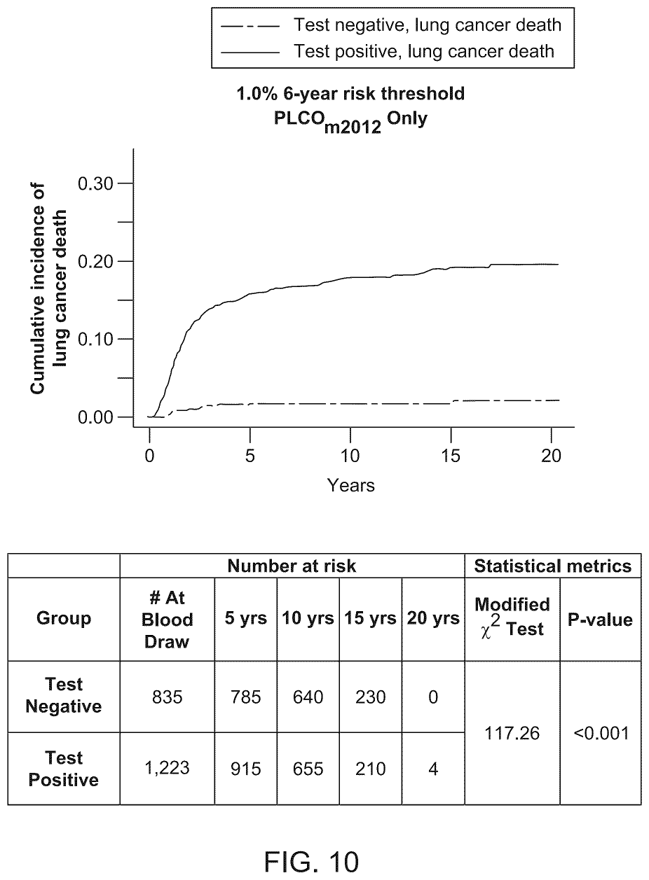

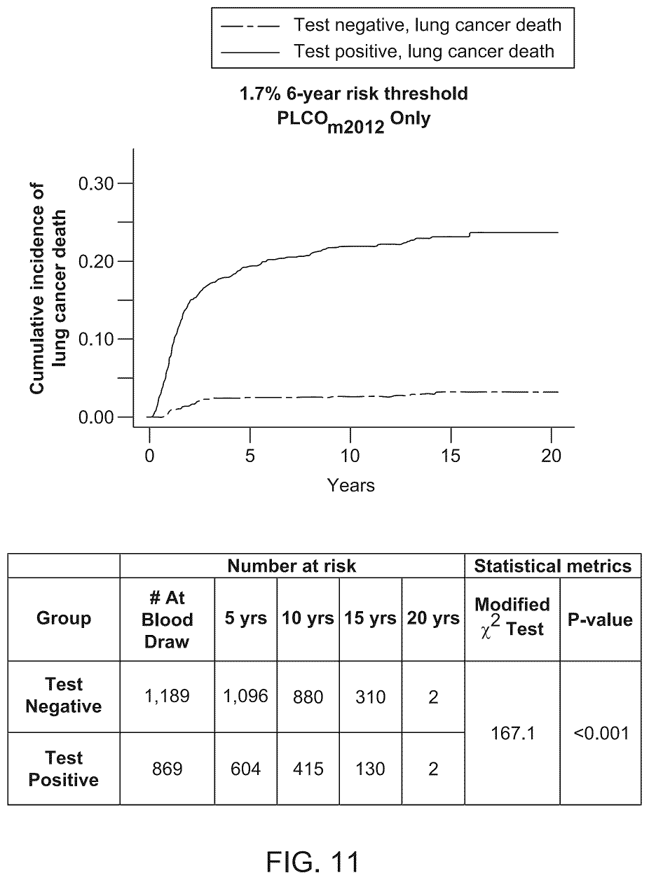

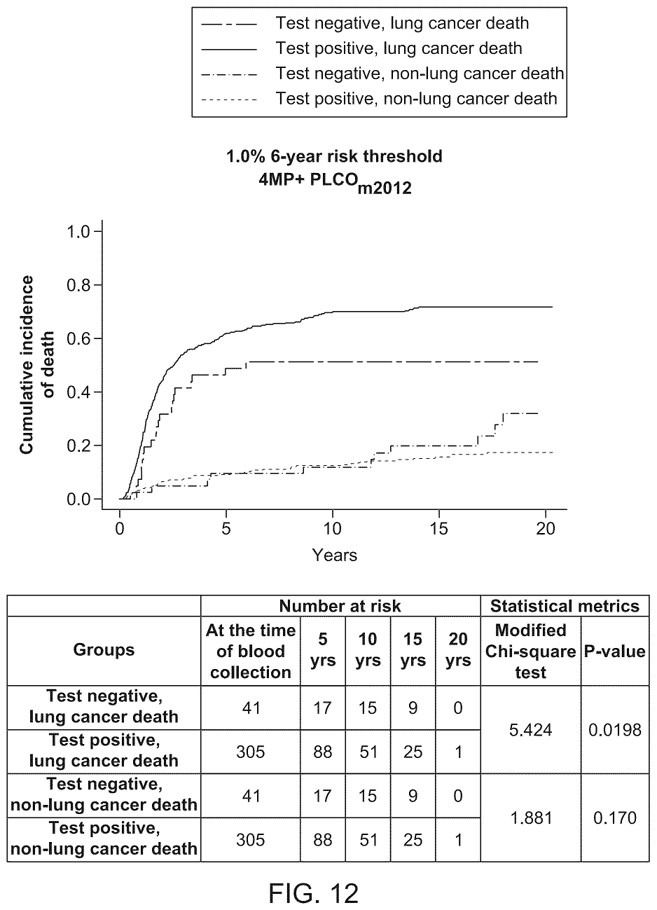

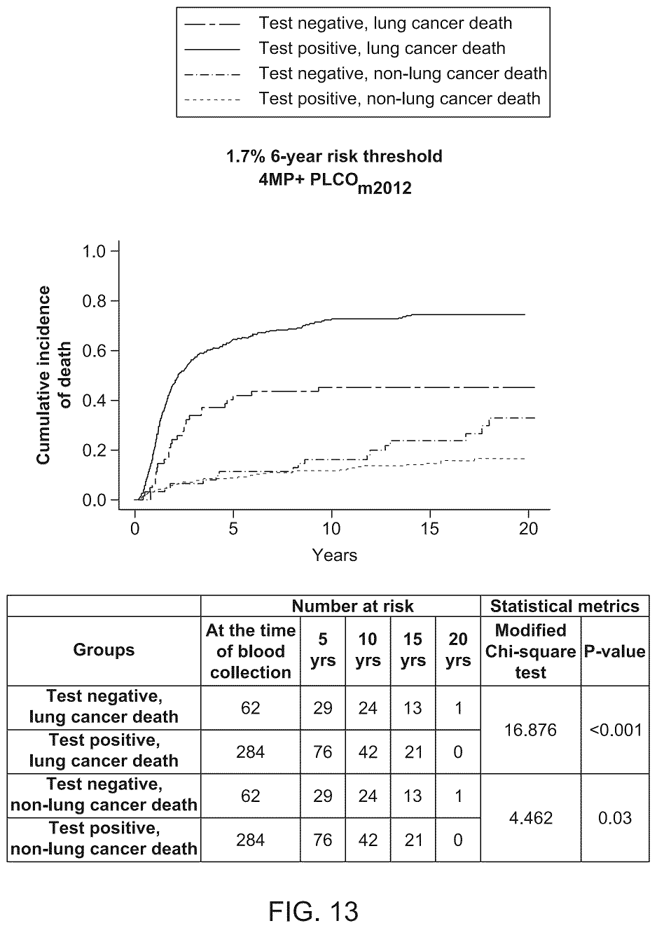

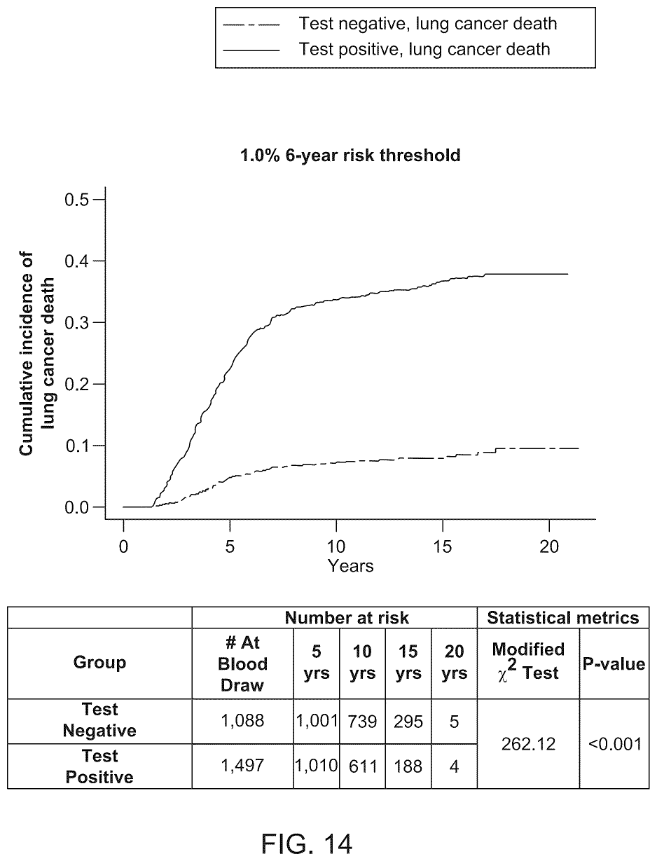

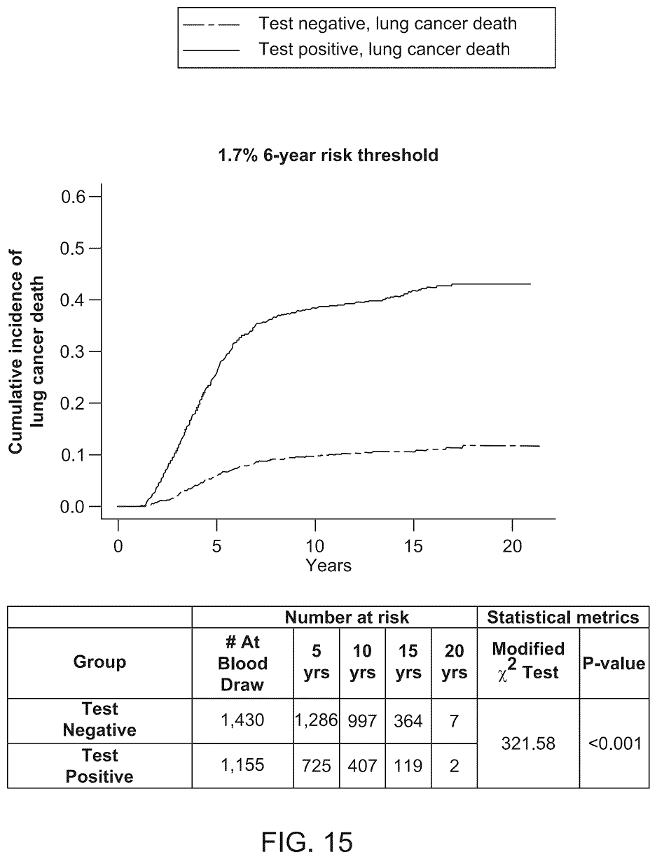

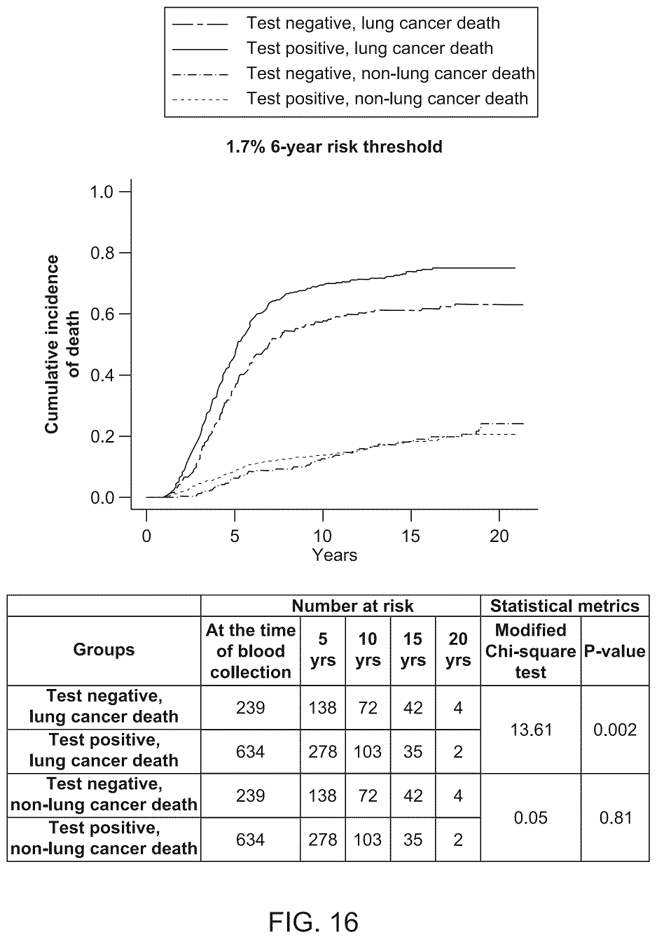

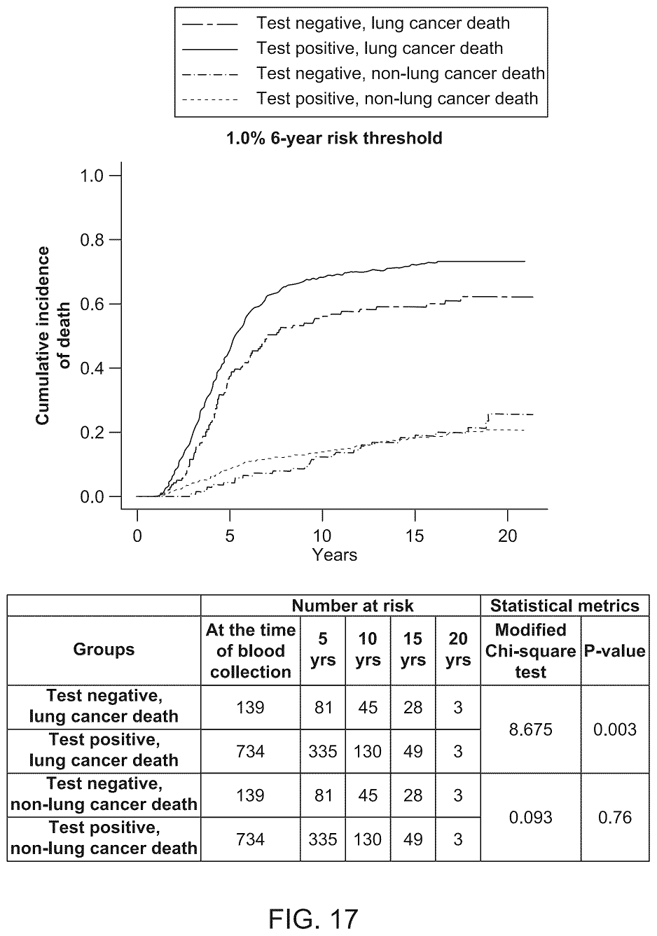

When considering the 1.7% 6-year risk threshold, compared to test-negative PLCO individuals (n=1,253), the cumulative incidence of lung cancer death was statistically significantly higher in test-positive cases (n=805) (modified χ²: 277.04, P<0.0001) with respective sub-distributional and lung cancer death specific hazard ratios of 12.82 (95% CI: 8.67-18.77) and 17.08 (95% Cl: 9.61-10.64). Compared to test-negative cases (n=990), at the 1.0% 6-year risk threshold, test positive cases (n=1,068) had a statistically significantly higher cumulative incidence of lung cancer death (modified χ²: 166.27, P: <0.001) with corresponding sub-distributional and lung cancer death specific hazard ratios of 9.88 (95% CI: 6.44-15.18) and 10.65 (95% CI: 6.93-16.37), respectively. (FIGS. 7-17; Tables 11-14).

TABLE 11
Sub-distributional hazard model and cause-specific hazard model ratios for individuals
with ≥10 PY smoking history at 1.0% and 1.7% 6-year risk thresholds.

| | | | Sub-distributional hazard model | | | | Cause-specific hazard model | | | |
|---|---|---|---|---|---|---|---|---|---|---|
| | | | Lung cancer | | Death from | | Lung cancer | | Death from | |
| | | | death | | other causes | | death | | other causes | |
| Risk | | Number at risk | Hazard | p- | Hazard | p- | Hazard | p- | Hazard | p- |
| threshold | Criteria | at baseline‡¥ | ratio | value | ratio | value | ratio | value | ratio | value |
| 1.7% | USPSTF2013 | Test Positive = 978 | 3.78 | <0.001 | 1.60 | <0.001 | 3.93 | <0.001 | 1.94 | <0.001 |
| risk | | Test Negative = 1,083 | (2.82-5.06) | | (1.34-1.89) | | (2.93-5.26) | | (1.63-2.31) | |
| threshold | PLCOₘ₂₀₁₂ | Test Positive = 869 | 6.25 | <0.001 | 2.1 | <0.001 | 6.69 | <0.001 | 2.74 | <0.001 |
| | | Test Negative = 1,189 | (4.56-8.57) | | (1.77-2.49) | | (4.88-9.17) | | (2.30-3.26) | |
| | 4MP | Test Positive = 926 | 8.78 | <0.001 | 1.64 | <0.001 | 9.39 | <0.001 | 2.14 | <0.001 |
| | | Test Negative = 1,132 | (6.08-12.66) | | (1.38-1.94) | | (6.51-13.55) | | (1.80-2.54) | |
| | 4MP + | Test Positive = 805 | 12.82 | <0.001 | 2.22 | <0.001 | 14.08 | <0.001 | 3.18 | <0.001 |
| | PLCOₘ₂₀₁₂ | Test Negative = 1,253 | (8.76-18.77) | | (1.87-2.62) | | (9.61-20.64) | | (2.67-3.78) | |
| 1.0% | USPSTF2021 | Test Positive = 1,118 | 4.27 | <0.001 | 1.46 | <0.001 | 4.41 | <0.001 | 1.76 | <0.001 |
| risk | | Test Negative = 943 | (3.07-5.94) | | (1.23-1.74) | | (3.18-6.14) | | (1.47-2.09) | |
| threshold | PLCOₘ₂₀₁₂ | Test Positive = 1,223 | 7.91 | <0.001 | 2.50 | <0.001 | 8.40 | <0.001 | 3.08 | <0.001 |
| | | Test Negative = 835 | (5.06-12.33) | | (2.05-3.06) | | (5.37-13.14) | | (2.52-3.77) | |
| | 4MP | Test Positive = 1,236 | 11.15 | <0.001 | 1.94 | <0.001 | 11.82 | <0.001 | 2.40 | <0.001 |
| | | Test Negative = 822 | (6.61-18.79) | | (1.61-2.34) | | (7.01-19.93) | | (1.98-2.91) | |
| | 4MP + | Test Positive = 1,068 | 9.88 | <0.001 | 2.62 | <0.001 | 10.65 | <0.001 | 3.37 | <0.001 |
| | PLCOₘ₂₀₁₂ | Test Negative = 990 | (6.44-15.18) | | (2.17-3.15) | | (6.93-16.37) | | (2.78-4.07) | |

Abbreviations: 4MP- four marker protein panel; PLCO- Prostate Lung Colorectal and Ovarian; USPSTF- United States Preventative Services Task Force.
‡Test-positive cases are defined as those individuals with 4MP, PLCOₘ₂₀₁₂, or 4MP + PLCOₘ₂₀₁₂ scores >1.7% and 1.0% 6-year risk thresholds. Test-negative are cases are those with scores ≤1.7% and 1.0% 6-year risk thresholds.
¥42 individuals lacked sufficient information to calculate PLCOₘ₂₀₁₂.

TABLE 12
Sub-distributional hazard model and cause-specific hazard model ratios for lung cancer
cases diagnosed within 1 year of blood draw at 1.7% and 1.0% 6-year risk thresholds.

| | | | Sub-distributional hazard model | | | | Cause-specific hazard model | | | |
|---|---|---|---|---|---|---|---|---|---|---|
| | | | Lung cancer | | Death from | | Lung cancer | | Death from | |
| | | | death | | other causes | | death | | other causes | |
| Risk | | Number at risk | Hazard | p- | Hazard | p- | Hazard | p- | Hazard | p- |
| threshold | Criteria | at baseline‡ | ratio | value | ratio | value | ratio | value | ratio | value |
| 1.7% | USPSTF2013 | TP = 250 | 1.40 | 0.031 | 0.65 | 0.1 | 1.40 | 0.029 | 0.91 | 0.726 |
| risk | | TN = 96 | (1.03-1.91) | | (0.39-1.09) | | (1.04-1.89) | | (0.54-1.55) | |
| threshold | PLCOₘ₂₀₁₂ | TP = 269 | 1.50 | 0.016 | 0.67 | 0.14 | 1.56 | 0.008 | 0.99 | 0.956 |
| | | TN = 77 | (1.08-2.09) | | (0.39-1.14) | | (1.12-2.16) | | (0.57-1.72) | |
| | 4MP | TP = 285 | 1.89 | 0.001 | 0.59 | 0.057 | 2.06 | <0.001 | 0.98 | 0.945 |
| | | TN = 61 | (1.28-2.77) | | (0.34-1.02) | | (1.40-3.03) | | (0.55-1.74) | |
| | 4MP + | TP = 284 | 2.23 | <0.001 | 1.13 | 0.68 | 2.36 | <0.001 | 1.03 | 0.925 |
| | PLCOₘ₂₀₁₂ | TN = 62 | (1.75-3.89) | | (0.63-2.04) | | (1.59-3.50) | | (0.58-1.82) | |
| 1.0% | USPSTF2021 | TP = 275 | 1.32 | 0.13 | 0.64 | 0.12 | 1.31 | 0.117 | 0.87 | 0.62 |
| risk | | TN = 71 | (0.92-1.89) | | (0.37-1.12) | | (0.94-1.83) | | (0.49-1.53) | |
| threshold | PLCOₘ₂₀₁₂ | TP = 312 | 1.32 | 0.25 | 0.85 | 0.67 | 1.36 | 0.197 | 1.22 | 0.622 |
| | | TN = 34 | (0.82-2.11) | | (0.39-1.83) | | (0.86-2.15) | | (0.55-2.71) | |
| | 4MP | TP = 311 | 2.40 | 0.002 | 0.49 | 0.024 | 2.52 | 0.001 | 0.92 | 0.8 |
| | | TN = 35 | (1.38-4.18) | | (0.26-0.91) | | (1.47-4.33) | | (0.47-1.78) | |
| | 4MP + | TP = 305 | 1.69 | 0.024 | 0.61 | 0.12 | 1.80 | 0.01 | 1.00 | 0.995 |
| | PLCOₘ₂₀₁₂ | TN = 41 | (1.07-2.66) | | (0.33-1.13) | | (1.15-2.82) | | (0.52-1.94) | |

Abbreviations: 4MP- four marker protein panel; PLCO- Prostate Lung Colorectal and Ovarian; USPSTF- United States Preventative Services Task Force.
‡Test-positive (TP) cases are defined as those individuals with 4MP, PLCOₘ₂₀₁₂, or 4MP + PLCOₘ₂₀₁₂ scores >1.7% and 1.0% 6-year risk thresholds. Test-negative (TN) are cases are those with scores ≤1.7% and 1.0% 6-year risk thresholds.

TABLE 13
Sub-distributional hazard model and cause-specific hazard model ratios for lung cancer
cases diagnosed within 1-6 years of blood draw at 1.7% and 1.0% 6-year risk thresholds.

| | | | Sub-distributional hazard model | | | | Cause-specific hazard model | | | |
|---|---|---|---|---|---|---|---|---|---|---|
| | | | Lung cancer | | Death from | | Lung cancer | | Death from | |
| | | | death | | other causes | | death | | other causes | |
| Risk | | Number at risk | Hazard | p- | Hazard | p- | Hazard | p- | Hazard | p- |
| threshold | Criteria | at baseline‡ | ratio | value | ratio | value | ratio | value | ratio | value |
| 1.7% | USPSTF2013 | TP = 1,252 | 2.28 | <0.001 | 1.51 | <0.001 | 2.46 | <0.001 | 1.94 | <0.001 |
| risk | | TN = 1083 | (1.93-2.71) | | (1.28-1.77) | | (2.07-2.91) | | (1.65-2.82) | |
| threshold | PLCOₘ₂₀₁₂ | TP = 1,244 | 3.86 | <0.001 | 1.84 | <0.001 | 4.30 | <0.001 | 2.73 | <0.001 |
| | | TN = 1,343 | (3.21-4.62) | | (1.57-2.16) | | (3.58-5.16) | | (2.32-3.20) | |
| | 4MP | TP = 1.254 | 3.08 | <0.001 | 1.52 | <0.001 | 3.41 | <0.001 | 2.2 | <0.001 |
| | | TN = 1,331 | (2.59-3.66) | | (1.30-1.78) | | (2.86-4.06) | | (1.80-2.47) | |
| | 4MP + | TP = 1,155 | 4.69 | <0.001 | 1.86 | <0.001 | 5.39 | <0.001 | 3.00 | <0.001 |
| | PLCOₘ₂₀₁₂ | TN = 1,430 | (3.90-5.62) | | (1.59-2.17) | | (4.48-6.47) | | (2.56-3.53) | |
| 1.0% | USPSTF2021 | TP = 1,503 | 2.60 | <0.001 | 1.33 | <0.001 | 2.77 | <0.001 | 1.73 | <0.001 |
| risk | | TN = 1,082 | (2.16-3.13) | | (1.13-1.56) | | (2.29-3.27) | | (1.47-2.03) | |
| threshold | PLCOₘ₂₀₁₂ | TP = 1,695 | 5.58 | <0.001 | 2.14 | <0.001 | 6.18 | <0.001 | 3.02 | <0.001 |
| | | TN = 890 | (4.28-7.27) | | (1.77-2.58) | | (4.74-8.05) | | (2.49-3.66) | |
| | 4MP | TP = 1,655 | 4.16 | <0.001 | 1.63 | <0.001 | 4.57 | <0.001 | 2.23 | <0.001 |
| | | TN = 930 | (3.31-5.22) | | (1.36-1.93) | | (3.62-5.74) | | (1.87-2.67) | |
| | 4MP + | TP = 1,497 | 5.27 | <0.001 | 2.17 | <0.001 | 5.96 | <0.001 | 3.22 | <0.001 |
| | PLCOₘ₂₀₁₂ | TN = 1,088 | (4.21-6.61) | | (1.82-2.58) | | (4.75-7.48) | | (2.69-3.84) | |

Abbreviations: 4MP- four marker protein panel; PLCO- Prostate Lung Colorectal and Ovarian; USPSTF- United States Preventative Services Task Force.
‡Test-positive (TP) cases are defined as those individuals with 4MP, PLCOₘ₂₀₁₂, or 4MP + PLCOₘ₂₀₁₂ scores >1.7% and 1.0% 6-year risk thresholds. Test-negative (TN) are cases are those with scores ≤1.7% and 1.0% 6-year risk thresholds.

TABLE 14
Survival time amongst lung-cancer case diagnosed within
one year of blood draw stratified based on 4MP +
PLCOm2012 scores > or ≤1.7% and 1.0% 6-year risk thresholds.

| | | | Median survival time (years) in |
|---|---|---|---|
| 6-year Risk | | Number at risk | individuals died from lung cancer |
| Threshold | Group† | at baseline | Median (1st-3rd quantile) |
| 1.7% | Test-positive | 284 | 2.19 (2.05-2.40) |
| | Test-negative | 62 | 4.97 (4.60-5.93) |
| 1.0% | Test-positive | 305 | 2.52 (2.20-2.69) |
| | Test-negative | 41 | 3.38 (2.53-4.97) |

†Test-positive cases are defined as those individuals with 4MP + PLCOₘ₂₀₁₂ scores >1.7% and 1.0% 6-year risk thresholds. Test-negative are cases are those with 4MP + PLCOm2012 scores ≤1.7% and 1.0% 6-year risk thresholds.

The 4MP+PLCOₘ₂₀₁₂ better predicts lung cancer specific mortality compared to USPSFT criteria, yielding improvements in sensitivity, specificity, and PPV. In the PLCO cohort, the LCDRAT model had a reported AUC of 0.81 (95% CI: 0.79-0.83) for predicting lung cancer death amongst those with smoking history. In comparison, the 4MP+PLCOₘ₂₀₁₂ model yielded an AUC of 0.88 (95% CI: 0.86-0.90) for predicting lung cancer-specific mortality among ever-smoker individuals.

Testing of the 4MP would be useful for individuals who are currently eligible for LDCT screening and expanded to additionally include individuals who have ≥10 PY smoking history. Individuals identified to be at high-risk of lung cancer incidence or death, based on 4MP+PLCOₘ₂₀₁₂ scores≥1.0% 6-year risk, corresponding to USPSF2021 criteria, would be referred to LDCT through shared decision making. Uptake to lung cancer screening programs, even for those eligible, has stubbornly remained below 15%, and a positive biomarker test may act as additional impetus for eligible individuals to undergo screening. For those individuals that lack sufficient information required for PLCOₘ₂₀₁₂, the 4MP alone may be used to inform on the need for LDCT based on the individual's risk profile. Testing of 4MP should be performed regularly with testing intervals matching their degree of risk. For countries outside of the United States that have not yet adopted USPSFT2021 criteria or that have not implemented LCS, the improved performance of the 4MP+PLCOₘ₂₀₁₂ at the more stringent decision-making threshold of ≥1.7% 6-year risk may select for individuals at exceptionally high-risk of lung cancer death that would benefit from LDCT while limiting the number of false-positives associated with a lower risk threshold.

The blood-based 4MP biomarker panel in combination with the PLCOₘ₂₀₁₂ model offers improved means for individualized risk assessment for lethal lung cancers, as compared to current USPSTF criteria and identifies individuals at high risk of a lethal lung cancer. The test has potential to better select for individuals who would benefit from LDCT screening.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of predicting the prognosis of an individual having a lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
wherein the combined model score indicates the prognosis of the individual with respect to time to progression and overall survival.

2. The method of claim 1, wherein the individual is predicted as having a poor lung cancer survival prognosis when the combined model score is above a pre-defined risk threshold.

3. A method of predicting disease outcome in an individual having lung cancer, comprising:
determining a combined model score using a logistic regression with the PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having an increased risk of a poor disease outcome when the combined model score is above is above a pre-defined risk threshold.

4. A method for identifying an individual at high risk of lung cancer death, comprising:
determining a combined model score using a logistic regression with a PLCOₘ₂₀₁₂ risk model score and a biomarker score, wherein the biomarker score measures the levels of biomarkers CEA, CA125, CYFRA21-1, and Pro-SFTPB in a biological sample obtained from the patient; and
identifying the individual as having a high risk of lung cancer death when the combined model score is above is above a pre-defined risk threshold.

5. The method of any one of the preceding claims, wherein the PLCOₘ₂₀₁₂ risk model score is based on baseline questionnaire information comprising age, race or ethnic group, education, body mass index, chronic obstructive pulmonary disease, personal history of cancer, family history of lung cancer and smoking status (current v former), intensity, duration, and quit time.

6. The method of any one of the preceding claims, wherein the combined model score is calculated with the equation: −11.836+1.6160*(0.4730*log[CA125]+0.6531*log[CEA]+0.2612*log[CYFRA21-1]+0.9238*log[Pro-SFTPB])+0.9861*(PLCOₘ₂₀₁₂ score).

7. The method of any one of the preceding claims, wherein the pre-defined risk threshold is the 1.0% 6-year risk threshold.

8. The method of claim 7, wherein a combined model score greater than −4.595 is considered a positive test.

9. The method of any one of claims 1-6, wherein the pre-defined risk threshold is the 1.7% 6-year risk threshold.

10. The method of claim 9, wherein a combined model score is greater than −4.057 is considered a positive test.

11. The method of any one of the preceding claims, wherein the individual is asymptomatic.

12. The method of any one of the preceding claims, wherein the individual is high-risk.

13. The method of any one of the preceding claims, further comprising calculating the life span of the individual.

14. The method of any one of the preceding claims, wherein the levels of biomarkers CEA, CA125, CYFRA21-1, and Pro-SFTPB in a biological sample obtained from the individual are determined by an immunoassay.

15. The method of any one of the preceding claims, wherein each of the biomarkers CEA, CA125, CYFRA21-1, and Pro-SFTPB in a biological sample obtained from the individual generates a detectable signal.

16. The method of claim 15, wherein the detectable signals are detectable by a spectrometric method.

17. The method of claim 16, wherein the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

18. The method of claim 17, wherein the spectrometric method is mass spectrometry.

19. The method of claim 18, wherein the mass spectrometry is LC-TOF-MS.

20. The method of any one of the preceding claims, wherein the lung cancer is early stage (e.g., stage I or II).

21. The method of any one of claims 1-20, wherein the lung cancer is advanced stage (e.g., stage III or IV).

22. The method of any one of the preceding claims, wherein the individual has a smoking history of ≥10 pack years.

23. The method of any one of the preceding claims, wherein the individual is between the age of 50 and 80 years.

24. The method of any one of the preceding claims, wherein the AUC of the method is greater than the AUC for a different biomarker, biomarkers, panel, assay, algorithm, model, or any combination thereof.

25. The method of claim 24, wherein the AUC is greater than 0.85.

26. The method of claim 25, wherein the AUC is between 0.86 and 0.90.

27. The method of claim 26, wherein the AUC is about 0.88.

28. The method of any one of claims 1-23, wherein the sensitivity and specificity values at a ≥1.7%/6-year risk threshold of the method are greater than the sensitivity and specificity values for a different biomarker, biomarkers, panel, assay, algorithm, model or any combination thereof.

29. The method of claim 28, wherein the sensitivity is greater than 0.80 and the specificity is greater than 0.65.

30. The method of claim 29, wherein the sensitivity is between 0.82 and 0.91 and the specificity is between 0.70 and 0.72.

31. The method of claim 30, wherein the sensitivity is about 0.85 and the specificity is about 0.71.

32. The method of any one of claims 1-23, wherein the sensitivity and specificity values at a ≥1.0%/6-year risk threshold of the method are greater than the sensitivity and specificity values for a different biomarker, biomarkers, panel, assay, algorithm, model or any combination thereof.

33. The method of claim 32, wherein the sensitivity is greater than 0.85 and the specificity is greater than 0.55.

34. The method of claim 33, wherein the sensitivity is between 0.87 and 0.94 and the specificity is between 0.56 and 0.59.

35. The method of claim 34, wherein the sensitivity is about 0.90 and the specificity is about 0.58.

36. The method of any one of the preceding claims, wherein the individual is subsequently designated for further lung cancer screening or treatment.

37. The method of claim 36, wherein the screening is chosen from endoscopic ultrasound, magnetic resonance imaging (MRI), and computed topography (CT) scans.

38. The method of claim 37, wherein the screening is performed annually.

39. The method of claim 37, wherein the screening is performed semi-annually.

40. The method of claim 36, wherein the treatment is chosen from surgery, chemotherapy, immunotherapy, radiation therapy, targeted therapy, or a combination thereof.
